(19) United States
(12) Patent Application Publication
(10) Pub. No.: US 2013/0197340 A1
(43) Pub. Date: Aug. 1, 2013

(54) Potential Artery-to-Vein Disease State Detection

(71) Applicant: University of Washington through its Center for Commercialization, Seattle, WA (US)

(72) Inventors: Joan E. Sanders, Sammamish, WA (US); Christian Redd, Seattle, WA (US)

(73) Assignee: University of Washington through its Center for Commercialization, Seattle, WA (US)

(21) Appl. No.: 13/735,755

(22) Filed: Jan. 7, 2013

Related U.S. Application Data
(60) Provisional application No. 61/583,934, filed on Jan. 6, 2012.
(60) Provisional application No. 61/682,582, filed on Aug. 13, 2012.

(51) Int. Cl. A61B 5/02; A61B 5/00

(52) U.S. Cl. CPC A61B 5/02007; A61B 5/4836; A61B 5/4519; A61B 5/6828; A61B 5/7275
USPC 600/384

(57) ABSTRACT

A method/apparatus/system for detection of a potential disease state is disclosed. A pair of electrodes is attached to a limb and the electrodes are energized. The energizing of the electrodes creates a first signal in the form of current data. If the first signal is consistent with a potential disease state, including a potential artery-to-vein disease state, an indication of the potential disease state is provided.

### CROSS-REFERENCES TO RELATED APPLICATIONS

This application is a non-provisional application, and claims the benefit of U.S. Provisional Application No. 61/583,934 filed on Jan. 6, 2012, and U.S. Provisional Application No. 61/682,582 filed on Aug. 13, 2012, both of which are hereby incorporated by reference in their entireties herein.

### STATEMENT AS TO RIGHTS TO INVENTIONS MADE UNDER FEDERALLY SPONSORED RESEARCH AND DEVELOPMENT

NOT APPLICABLE

### BACKGROUND OF THE INVENTION

There are a variety of challenges associated with identification and treatment of circulatory and/or vascular disease states, and particularly with a variety of disease states associated with artery-to-vein transport dysfunction, including compartment syndrome, vascular trauma, chronic venous insufficiency, and/or the like.

Compartment syndrome is an elevation of the interstitial pressure in a closed fascial compartment that results in microvascular compromise. Compartment syndromes can be classified as both acute and chronic, with different underlying causes for each. Compartment syndromes can develop in a variety of places, but are most commonly found in the anterior and deep compartments of the leg. Acute compartment syndromes typically result from orthopedic or vascular trauma, but can also occur during or following acute ischemic events. The most common cause of an acute compartment syndrome is a bony fracture, especially the tibia. Acute compartment syndrome also will often be a result of blunt trauma, as a result of musculoskeletal injury or direct tissue damage. Chronic compartment syndromes have different causes than acute compartment syndromes, most often as a result of increased compartment pressures during repetitive vigorous exertion. During vigorous exercise, muscle volume can increase by 20%, causing an increase in pressure within the fixed compartment. This is often manifested in long-distance runners and military recruits and commonly involves the anterior or deep posterior compartment of the leg.

The proper diagnosis of compartment syndrome can be based on: clinical suspicion, mechanism/placement of the injury, a physical examination, and compartment pressure measurements. The clinical presentation is often subtle in the early stage; however, early diagnosis is critical for positive outcomes. Compartment syndrome diagnosis can be relatively straightforward with a cooperative and alert patient, while it is often very challenging in patients with an altered mental state. In this case, a compartment pressure measurement is incredibly valuable in the decision making process. The current standard for compartment pressure measurements may be the Stryker intracompartmental pressure monitoring system (Stryker Surgical, Kalamazoo, Mich.) that uses an 18 gauge needle inserted directly into the compartment to determine the internal pressure. Proposed noninvasive assays suitable to measure compartmental pressures tend to have a high specificity and low sensitivity, which may explain a lack of adoption of noninvasive diagnostic tools for these vascular diseases.

Following the diagnosis of a compartment syndrome, immediate and complete decompression should be sought to provide a positive outcome. The treatment of compartment syndrome involves a properly timed fasciotomy by an orthopedic surgeon. The exact procedure is location dependent, but can include: complete incision of the overlying skin, complete incision along the full length of the fascia, local wound care and debridement, and finally reestablishment of healthy skin coverage. During surgery, assessment of the completeness of fasciotomy is largely visual, with the surgeon responsible for determining that all involved compartments are adequately decompressed. Although a limb may contain several muscle compartments, and although some patients may benefit from decompression of only a subset (rather than all) of the compartments of an affected limb, the current standard of care includes performing fasciotomies on all muscle compartments of the affected limb. Positive outcomes are highly dependent on timely surgical intervention. Complication rates rise if the surgical procedure is delayed more than 12 hours. Almost half of patients with delayed (12 hr) fasciotomy may require major amputation and 92% may have significant neuropathy.

Diagnosis and/or treatment of a variety of other vascular disease states suffer from challenges that may be at least marginally related to those described above. For example, traumatic injury to the lower extremities can more generally result in damage to the vascular system, with varying degrees of manifestation and severity. In many cases, especially in the military theatre, extremity vascular trauma is often associated with multiple other injuries along with local bony and soft tissue defects. Management of multiple injury patients is often challenging, with restoring perfusion to an ischemic extremity preferably taking place within 6-8 hours so as to increase the probability of function being salvaged. Compartment syndrome is often a secondary result of vascular repair, usually associated with reperfusion injury and local hematoma formation. Vascular trauma can therefore be a significant contributor to both acute and chronic fluid transport issues, including those mentioned previously.

Diagnosis of vascular trauma is difficult, with many injuries being asymptomatic and only diagnosed during surgical exploration for other injuries. Arterial injuries are usually considered more critical when assigning treatment priorities, with venous injuries addressed as a secondary concern. In diagnosis of vascular injuries resulting from extremity trauma, hard signs of vascular injury may include the 6 Ps (Pain, Pallor, Pulselessness, Poikilothermia, Paresthesia, Paralysis), hypotension/anemia, and hematoma. Soft signs may include: small hematoma, minor bleeding, and wound in proximity to neurovascular bundle. In the case of soft signs, further measurement may be needed to determine the existence and extent of vascular trauma. It is common however, for venous injuries to be accompanied by arterial injuries; the diagnosis and treatment is often linked. In a recent study, Parry et al found 82.6% of serious lower extremity venous injuries were accompanied by arterial injuries. While acute cases are covered in depth due to their high rate of occurrence (reportedly 7% of total patients in Iraq and Afghanistan), there is a dearth of dependable research on the occurrence of delayed manifestations of venous injuries. This may be partly due to the difficulty in diagnosis of isolated venous injuries, which are often only discovered if massive swelling or life-threatening hemorrhage occurs.

In the past, the majority of vascular injuries were often treated with ligation. While advances in medical science allow for venous repair, there is still a certain level of controversy regarding the circumstances for ligation or repair. While the individual case may be left up to the discretion of the surgeon, the general consensus is to make prudent use of both methods. For patients who are close to hemodynamic collapse, ligation may be the correct choice. For stable patients with central venous injuries, it may be best to make a concentrated effort to reestablish blood flow. While it can be assumed that ligation is a good choice for more distal venous structures, fluid shifts can result and require fasciotomy and resuscitation. Because of the reliance on individual judgment for diagnosis and treatment, additional qualitative (and ideally quantitative) measurement techniques would provide surgeons with a valuable assessment tool for use in cases of acute, chronic, and secondary vascular trauma.

Still other vascular disease states present related challenges. Chronic venous insufficiency concerns the inability or failure to reduce venous pressure with exertion. Under healthy circumstances, the venous valves and muscular pumps of the lower extremity limit the total accumulation of blood in the lower extremity veins. Failure of these mechanisms can be a result of different types of peripheral venous insufficiency, with the end result being ambulatory venous hypertension. Chronic venous hypertension or sustained venous pressure elevation results in a variety of pathological defects and negative prognosis for the affected patient. Lower limb edema, change of pigmentation, and varicose veins are all common indicators of chronic venous insufficiency.

There are a variety of methods used to diagnose chronic venous insufficiency, making use of an examination by an attending physician as well as objective tests. Localized lower extremity edema and discoloration often support the diagnosis of venous hypertension. A full clinical history in addition to the physical examination is usually sufficient to diagnose severe clinical manifestations, but in more subtle cases, there are a variety of diagnostic tests that may be used. One commonly accepted standard for venous diagnostics is venography. This test can be used to provide a large amount of data regarding the hemodynamic and anatomic state of the venous system. Unfortunately, this diagnostic test is relatively expensive, invasive, and uncomfortable for the patients, and is therefore only used in specifically prescribed cases. Alternatively, air plethysmography (APG) can be used to measure venous volume and calculate a venous filling index. In the leg, the air displacement is measured within a polyurethane cuff that is wrapped around each limb and inflated to a pre-set pressure. APG has some drawbacks in that it is expensive, cannot precisely localize sites of venous reflux, and only provides an overall assessment of venous function. It also does not meet the precision standards desirable for a long term venous evaluation. Other diagnostic tests for venous function also exist, including: duplex ultrasonography (which may be even more common that venography), photoplethysmography, and the Ankle-Brachial Index.

The treatment of chronic venous insufficiency is variable and highly dependent on the history and background of the specific subject. Changes to the general diet and lifestyle of the patient, including a thorough review of the exercise regimen, is recommended in many cases. Subjects with edema resulting from fluid retention in the lower extremities can be prescribed a diuretic while being monitored for symptoms of intravascular volume depletion. In cases where an interventional procedure is deemed appropriate, different procedures are prescribed depending on the severity and location of the vascular issues. In cases of venous valvular incompetence, the current standard may be to apply less invasive endovascular treatment including chemical sclerotherapy, radiofrequency ablation, and laser therapy. In severe cases of venous outflow obstruction, surgical procedures may be appropriate and include venous ligation, subfascial endoscopic surgery, and valve reconstruction surgery.

Treatment of venous ailments is of particular importance for extremity injuries in a wartime theatre. Prehospital management of extremity wounds often occurs in deleterious circumstances with a minimum amount of available equipment. Because of the austere environment, field equipment can be lightweight, multipurpose, resilient, and effective. Another inherent difficulty in the management of battlefield wounds is the wide variety of injury modalities. Lower limb injuries can be a result of blast trauma, high-velocity gunshot wounds, blunt trauma, and shrapnel wounds and are often combined with other polytrauma sites on the injured subject. These factors combined result in different measurement, diagnosis, and treatment methods than those commonly used in clinical settings. For proper treatment of these ailments, there is a need for valuable, accurate, and portable devices to assist in the early diagnosis of lower limb vascular trauma.

The incidence of compartment syndrome and limbs at risk in combat casualties requiring evacuation has been estimated to be 15%. In Iraq and Afghanistan, severe extremity trauma caused by blast injuries has been a common presentation. In addition, many fractures that occur on the battlefield are open, which may be associated with a higher risk of vascular and soft-tissue injuries and subsequent compartment syndrome. Due to the austere environment, diagnosis of compartment syndrome is even more heavily weighted toward clinical evaluation than in the civilian setting. Compartmental pressure measurements may be used if available; however, they frequently do not enter into the treatment algorithm. In order to minimize the possibility of a false-negative evaluation of compartment syndrome, prophylactic fasciotomies may be required before any clinical sign of compartment syndrome. In addition, there is some initial data and speculation from deployed surgeons that suggests that decreased atmospheric pressure occurring during aeromedical evacuation might exacerbate muscle edema in the injured limb and contribute to or cause compartment syndrome. Better diagnosis of compartment syndrome may reduce the currently maintained low threshold for fasciotomy currently used to avoid the possibility of a missed diagnosis.

Vascular injuries on the battlefield present similar injury situations as compartment syndrome, and as such, are subject to the same limitations as battlefield compartment syndrome. Recognized vascular injuries should be definitively repaired before leaving the wartime theatre. These injuries are often addressed at the highest level trauma center available (in theatre: Level III). If the subject requires further care in a level V trauma center, average transport time may be several days. During the combat theatre in Iraq and Afghanistan (referred to as the Global War on Tenor, or GWOT) a significant portion of battle-related injuries include vascular injuries. Of these vascular injuries, the majority may be attributed to explosive fragmentation, and many from gunshot wounds. During combat operations in the GWOT, the DOD implemented a testing, training, and fielding program for battlefield tourniquets. The use of these tourniquets has been shown to increase survivability, with tourniquet use (absent shock) strongly correlated with subject survival. Because of this success, most casualties arriving with extremity vascular injuries are presented with applied prehospital tourniquets. Treatment of extremity vascular injuries can include temporary vascular shunting followed by venous repair (ligation or reattachment). Vascular injuries with delayed presentation occur frequently and have provided the basis for careful re-assessment strategies at each level of care. The standard for vascular screening in level III facilities can include contrast enhanced CTA scanning, which has been confirmed as a beneficial tool in the screening and diagnosis in soft sign vascular injury. While measurements, diagnosis, and treatment of vascular injury on the modern military battlefield have all been advanced, the greatest challenge may still be the simultaneous damage control and vascular construction that the surgeon is tasked with.

Because of the above, a device that aids in the monitoring and quantification of vascular injury severity while simultaneously reducing the demands on and/or responsibility of the surgeon and other attending medical care personnel would be a valuable tool in theatre, in a surgical setting, for first responders, and in a variety of other settings.

### BRIEF SUMMARY OF THE INVENTION

In some embodiments, the present disclosure provides methods for identifying a potential vascular disease state of a limb of a patient. The method can include placing a plurality of electrodes on the limb, including a first electrode on an outer surface of the limb adjacent a first region, and energizing at least some of the electrodes while the limb of the patient is in a first arterial to vein transport state so as to generate a first signal. The first signal can be used to determine the potential vascular disease state of the limb, and an indication of the potential disease state of the limb can be provided.

In some embodiments, the present disclosure provides a system that identifies a potential disease state of a limb of a patient. The system can include a plurality of electrodes (including a first limb surface electrode) and a controller. The controller can energize at least some of the electrodes so as to generate a first signal with the first electrode, determine that the first signal is consistent with a potential vascular disease state of the limb in response to the first signal, and provide an indication of the potential disease state of the limb.

In both the system and method aspects described herein, each region of the limb may include muscle and/or other tissue with associated veins and arteries. In many embodiments, a second electrode may be configured to be placed on the outer surface of the limb adjacent a second region of the limb, the second region including tissue with associated veins and arteries. The same (or alternatively, different) electrodes may be energized so as to generate a second signal with the second electrode, and the first signal and the second signal may be determined to correspond to the potential vascular disease state, with the potential vascular disease state comprising a disease state associated with artery-to-vein transport defects.

In exemplary embodiments, the potential vascular disease state can be localized as being present in the first region in response to the first and second signals. A therapy can be selectively directed to the first region so as to mitigate the potential vascular disease state. For example, the tissue of the first region may comprise muscle tissue of a first muscle compartment of the limb, and the tissue of the second region may comprise muscle tissue of a second muscle compartment of the limb. Fasciotomy may be selectively performed to the muscle tissue within the first muscle compartment while foregoing fasciotomy to the muscle tissue within the second muscle compartment, both in response to the localization of the potential vascular disease state within the first muscle compartment.

Optionally, information from the first signal is stored, with the information relating to the state of the region of the first muscle of the limb. The first electrode pair may be energized while the limb of the patient is in a second artery-to-vein transport state so as to generate a second signal, wherein the first signal is generated at a first time and the second signal is generated at a second time. A trend between the first signal and the second signal may be determined to be consistent with a potential artery-to-vein disease state of the limb.

In some cases, the first electrode pair may be included in an ambulatory bioimpedance system mounted to the patient during the first and second times. The first artery-to-vein transport state may comprise a relatively active exertion state, while the second artery-to-vein transport state comprises a relatively inactive exertion state. In these or other cases, the first artery-to-vein transport state may comprise a first orientation and the second artery-to-vein transport state comprises a different orientation. For example, when the limb comprises a leg the patient may be walking or running during the first time and may be resting (upright, sitting, prone, supine, or the like) during the second time (which may occur before or after the first time). Where the second state occurs after the first state the trend may be associated with post-exertion recovery. Advantageously, the ambulatory bioimpedance system described herein may be supported by the patient for over 1 hour (often being mounted to the patient for more than 2 hours, more than 4 hours, 8 hours or more, and/or a day or more) and the potential disease state can optionally be robustly identified using a plurality of trends among differing artery-to-vein transport states of the limb.

In many embodiments, the placing of the electrode pair can be performed in a field setting (such as by first responders, military medics, and/or the like) so that the first time is within 2 hours of a trauma to the patient. The second time may be after the first time by a post-trauma blood accumulation period so as to identify trauma-induced vascular diseases of the limb. These and other embodiments may be particularly beneficial for non-responsive patients. Embodiments of the systems and methods described herein may be employed before, during, and/or after vascular injury. For example, optional embodiments may be employed during surgery that is related or unrelated to the vascular disease state. Acute compartment syndrome and other vascular conditions may occur during surgical procedures, sometimes in the contralateral limb, due to the enforced positioning of the body and use of pressure cuffs. Vascular transport monitoring systems and method provided herein may be used with surgical procedures and positioning methods that might otherwise be seen as having significant risk as indicated by prior spontaneous development of compartment syndrome in an otherwise healthy limb.

The first electrode pair will often be included in a bioimpedance system having a plurality of electrode pairs configured for mounting to the limb in an associated plurality of locations. The bioimpedance system can apply a plurality of frequencies to the plurality of electrode pairs so as to generate a plurality of signals, and can determining the disease state from the plurality of signals.

Further areas of applicability of the present disclosure will become apparent from the detailed description provided hereinafter. It should be understood that the detailed description and specific examples, while indicating various embodiments, are intended for purposes of illustration only and are not intended to necessarily limit the scope of the disclosure.

### BRIEF DESCRIPTION OF THE DRAWINGS

The present disclosure is described in conjunction with the appended figures:

FIG. 1A is a schematic diagram illustrating an exemplary ambulatory embodiment of a bioimpedance system, along with its use for detection of potential vascular disease states;

FIG. 1B is a functional block diagram illustrating components of an exemplary system for detection of a potential vascular disease states;

FIG. 2 is block diagram showing further functional details of an exemplary device for detection of a potential disease state;

FIG. 3 is a schematic illustration of one embodiment of aspects of an exemplary device for detection of a potential disease state;

FIG. 4 is an illustration of one embodiment of electrode placement on a limb;

FIG. 5 is an illustration of one embodiment of radial electrode placement on a limb;

FIG. 6 is an illustration of one embodiment of longitudinal electrode placement on a limb;

FIG. 7 is an illustration of one embodiment of electrode and pressure cuff placement on a limb;

FIG. 8 is a flowchart depicting one embodiment of a process for performing a stress test;

FIG. 9 is a graph depicting impedance data collected during a stress test by electrodes on a limb;

FIG. 10 is a graph depicting volume data generated from impedance data collected during a stress test by electrodes on a limb;

FIGS. 11A and 11B are graphs illustrating the normalized outflow of a limb of a healthy individual and the normalized outflow of a limb of an unhealthy individual;

FIG. 12 is a flowchart illustrating one embodiment of a process for identifying a potential disease state of a limb of a patient;

FIG. 13 is a flowchart illustrating one embodiment of a process for localizing a potential disease state within a limb of a patient;

FIG. 14 is a flowchart illustrating one embodiment of a process for identifying a potential disease state of a limb of a patient over time;

FIGS. 15A and 15B schematically illustrating exemplary electrode structures for use in embodiments of the vascular disease state identification systems described herein;

FIG. 16 is a functional block diagram illustrating components included in an embodiment of an ambulatory processor that may be used in embodiments;

FIGS. 17A-17C present tabular experimental data as described herein;

FIGS. 18A-18C, 19A, and 19B present graphical experimental data as described herein.

In the appended figures, similar components and/or features may have the same reference label. Where the reference label is used in the specification, the description is applicable to any one of the similar components having the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION OF THE INVENTION

The ensuing description provides preferred exemplary embodiment(s) only, and is not intended to limit the scope, applicability or configuration of the disclosure. Rather, the ensuing description of the preferred exemplary embodiment(s) will provide those skilled in the art with an enabling description for implementing a preferred exemplary embodiment. It is understood that various changes may be made in the function and arrangement of elements without departing from the spirit and scope as set forth in the appended claims.

### Ambulatory Vascular Monitoring System

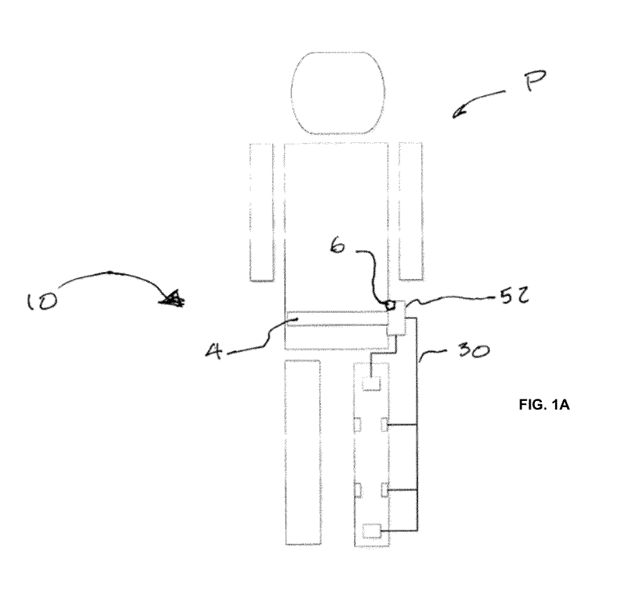

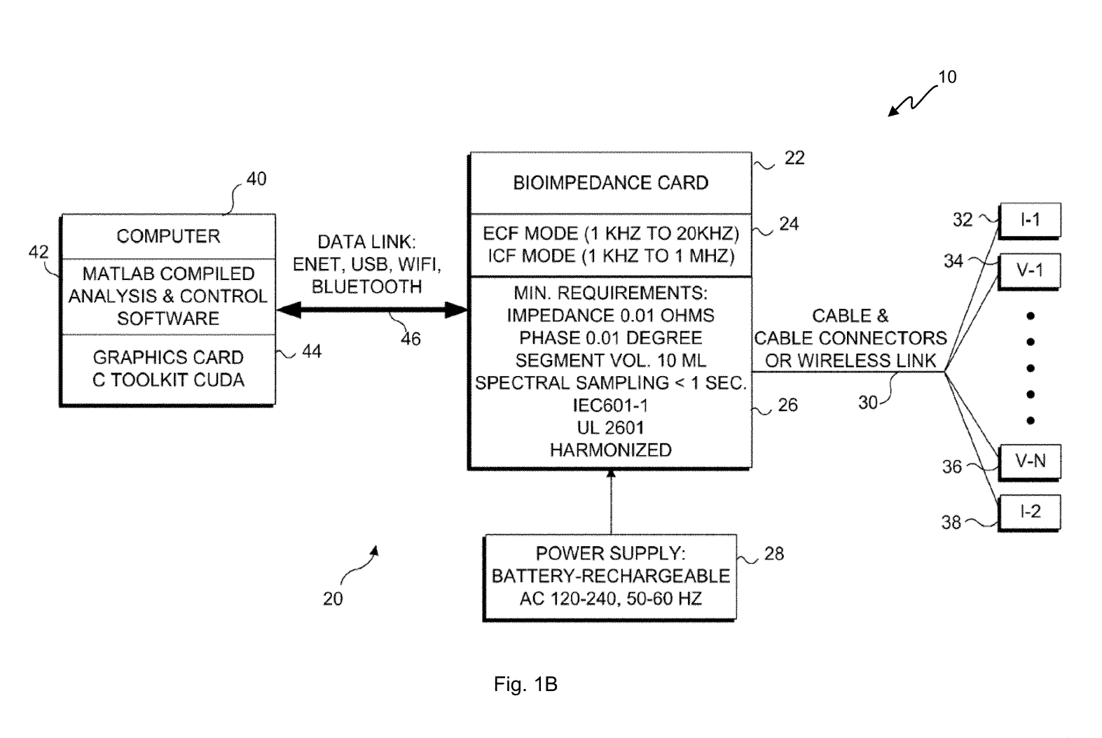

Referring now to FIG. 1A, an exemplary monitoring system 10 includes a controller 52 configured to be carried by and/or with a patient P. An array of electrodes may be coupled to controller 52 via leads 30, with the leads being affixed to target regions of a limb so that pairs of the electrodes are in electrical communication via differing muscle compartments of the limb. The electrodes and controller can be mounted to patient P via adhesive tape or substrates, a belt 4 or other support structure, a garment, or the like. Note that the electrodes may be included in a set of electrodes having adhesive substrates or other support structures that are configured to promote desired placement of pairs of the electrodes so as to facilitate monitoring of a plurality of compartments or regions of the leg (or other limb or limb portion). Systems may also be provided which are coupled to more than one limb.

Controller 52 includes a battery 6 or other power source to allow the system to gather data while patient P is untethered, thereby facilitating gathering of information regarding artery-to-vein transport while the patient is moved (such as on a stretcher or the like) and preferably while the patient is ambulatory. Such ambulatory and/or portable vascular monitoring may be particularly beneficial, as it may allow data to be gathered while the limb of the patient undergoes significant changes in its artery-to-vein transport state without interfering in transportation, care, and/or undesirably altering blood transport within the limb. For example, where an ambulatory system is configured to be affixed to the patient for more than an hour, such as when the ambulatory system is configured to gather data throughout a period of at least 2 hours, 4 hours, a half day, or a day or more, the patient will typically have periods of relatively high exertion while upright (such as during walking, jogging or running, performing household chores, and/or the like). These high-exertion upright periods may be proceeded and/or followed by upright, seated, and/or supine periods of more moderate sedentary activity, rest, or sleep. The transitory data obtained during associated changes in blood transport measurements of the differing regions of the limb can be combined with data relating to the orientation and/or movement of the patient (such as via an accelerometer or the like of controller 52), the heartbeat of the patient (via the limb-mounted electrodes, a dedicated heart monitoring electrode, or the like) and other data sources to provide robust information on the functionality of the vasculature of the limb. Alternatively, mounting of a portable vascular monitoring system to a trauma victim during triage and/or by first responders may allow the attending medical personnel to concentrate on more immediately life-threatening injuries, with the system monitors for and provides warning of a trend toward a potentially limb-threatening vascular disease state of one or more limbs.

### Bioimpedance Measurement

A bioimpedance measurement modality is used for the assessment of a variety of body parameters, and can be used, for example, to measure volumes and/or pressures within a body. Specifically, bioimpedance is a noninvasive means for assessing extracellular fluid (ECF) volume and intracellular fluid (ICF) volume within living tissue, and can be likewise used to non-invasively measure localized and/or general body pressures. ECF includes blood and interstitial fluid. Interstitial fluid is plasma that enters and leaves the interstitial space. Bioimpedance analysis has typically been used as a tool principally for body composition/body fat analysis and in the assessment of fluid imbalance in hemodialysis patients.

FIG. 1B depicts one embodiment of a potential disease state detection device 10. The potential disease state detection device 10 measures biological conductivity to detect the potential disease state, including, for example, a potential artery-to-vein disease state. Biological conductivity can occur through fat-free tissues and fluids, and is measured with bioimpedance. To conduct a bioimpedance test, a very low electrical current (<700 μA) is injected into the limb through current injecting electrodes 32, 38, while voltage potential is measured across voltage sensing electrodes 34, 36, as shown in FIG. 1. The electrodes 32, 34, 36, 38 are coupled through cables and cable connectors 30 to a bioimpedance controller 20. In some embodiments, some or all of the electrodes 32, 34, 36, 38 communicate with the bioimpedance controller 20 via a unique and/or shared channel, thereby allowing more specific control of, and data collection from the electrodes 32, 34, 36, 38. An alternative approach is to couple the electrodes 32, 34, 36, 38 to the controller using a wireless connection, such as Bluetooth, or WiFi (without any implied limitation). In this exemplary embodiment, the bioimpedance controller 20 includes a bioimpedance card 22, a control 24 to selectively operate in an ECF mode (by injecting current at a frequency range from 1 kHz to 20 kHz), in an ICF and ECF mode (by injecting current at a frequency range from 1 kHz to 1 MHz), or in a hybrid mode (by injecting current at frequency ranges from 1 kHz to 1 MHz). In some embodiments, current can be injected over a range of frequencies (between about 5 kHz and about 1 MHz) each second. Additionally, in some embodiments, the selection of the frequency of the current can affect the type of tissue penetrated by the current. For example, lower frequencies tend to travel through the ECF as cell membranes are not well-penetrated by low frequency signals. At high frequencies, however, current travels through both the ECF and ICF.

Data collected with combinations of currents at different frequencies can be used to calculate resistance of the tissue through which the current passed. In some embodiments, the data collected with combinations of currents at different frequencies can be used to calculate resistance of the ECF and/or ICF.

An alternative to the cable and cable connectors 30 is a wireless connection between the electrodes 32, 34, 36, 38 and bioimpedance electronics. A wireless connection offers to the user the capability of gathering bioimpedance data without the risks and hassles associated with wire connection. In one embodiment, current sensing electrodes 34, 36, which will be discussed in greater detail below, are configured for wireless use. To achieve a wireless connection to the current sensing electrodes 34, 36, small voltage sensing electrodes, amplifiers, power receiver, and data transmitter are embedded within a flexible non-conductive pad attached to the skin. The electronic components in the pad can communicate wirelessly with a nearby inductive power source and a data receiver that attaches to the bioimpedance card. A block 26 indicates that the minimum impedance required for the measurement circuit is 0.01 ohms, the phase is 0.01 degrees, the segment volume (i.e., the segment between voltage electrodes) is about 10 ml, and the spectral sampling occurs in less than 1 second. A power supply comprising rechargeable batteries 28 provides the power for the bioimpedance controller. A data link 46 comprising either an Ethernet cable, a universal serial bus (USB) link, or a wireless radio frequency link, e.g., a WiFi signal or a Bluetooth signal, conveys data related to the change in volume of the limb being monitored to a computer 40 that is running MatLab™ compiled analysis and control software 42, and which includes a graphics card with C toolkit Compute Unified Device Architecture (CUDA) 44. Alternative custom or commercially available computer software languages and tools may be employed for the analysis and control software 42, the graphical interface, and the like. The computer can display real-time changes in the ECF or ICF volume of a limb in the form of a graph (like those discussed below) during different activities of the subject, such as standing and walking.

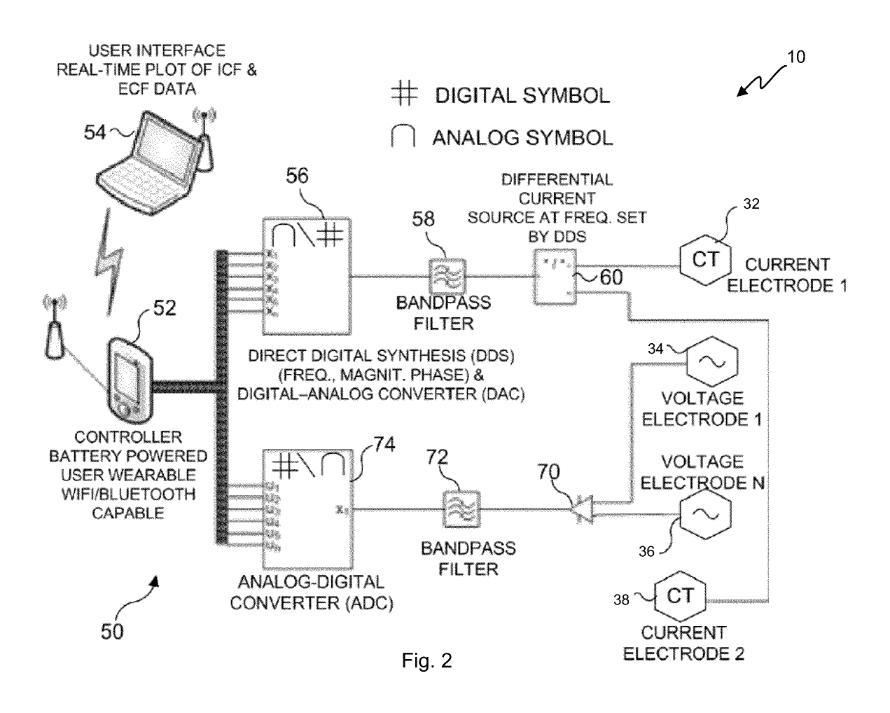

FIG. 2 illustrates further details of an exemplary bioimpedance measurement system 50, which includes a controller 52 that is battery powered and sufficiently small to be user wearable and which can communicate wirelessly (using WiFi or Bluetooth) with a laptop 54 that displays the real-time ICF and ECF volumetric change data or body pressure data. While not separately shown, controller 52 can include a storage such as a memory chip or card on which volumetric change data for a period of time (e.g., for 24 hours or more) can be stored, so that the data can be uploaded via the wireless communication on demand when desired. Controller 52 is coupled to a direct digital synthesis (DDS) card 56 with control of frequency, magnitude, and phase, and which includes a digital-analog converter (DAC—not separately shown). Current signals at the desired frequencies are applied to a bandpass filter 58, which passes a desired band of frequencies to a differential current source 60, at the frequency set by the DDS. This current is injected into the limb of the subject by current injection electrodes 32, 38. Current sensing electrodes 34, 36 are applied to the limb of the subject, and the differential voltage sensed between two adjacent current sensing electrodes 34, 36 and/or between one of the current injecting electrodes 32, 38 and one of the current sensing electrodes 32, 38 is applied to a differential amplifier 70, which produces a signal that is input to a bandpass filter 72. The resulting filtered signal is input to an analog-digital converter (ADC) 74, which converts the analog voltage level to a corresponding digital signal that is input to controller 52 for processing to determine volume change, and to thereby determine the bioimpedance.

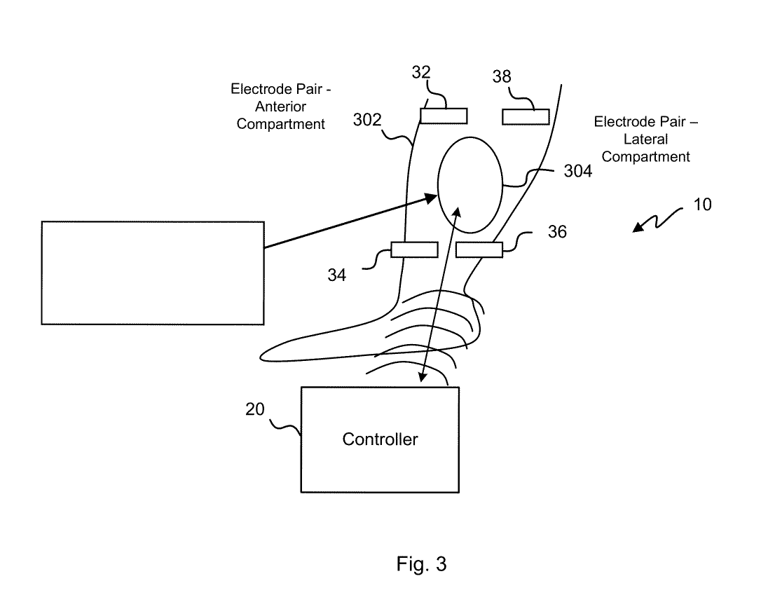

FIG. 3 is a schematic illustration of one embodiment of aspects of the potential disease state detection device 10. As seen in FIG. 3, the potential disease state detection device 10 can be used for the detection of a potential disease state in, for example, a limb 302 including a leg, an arm, or any part of a leg or arm.

The potential disease state detection device 10 depicted in FIG. 3 includes a pair of current injecting electrodes 32, 38 and a pair of current sensing electrodes 34, 36, although more or fewer current injecting and current sensing electrodes 32, 34, 36, 38 can be part of the potential disease state detection device 10.

The current injecting electrodes 32, 38 can provide electrical current to the limb 302. As such, the current injecting electrodes 32, 38 can comprise a conductive material configured to inject current into the limb 302, connection points configured to allow connection of the current injecting electrodes 32, 38 to a power source and/or measurement source, and features configured to attach the current injecting electrodes 32, 38 to the limb 302.

The current injecting electrodes 32, 38 can comprise a variety of shapes and sizes. In some embodiments, one or both of the current injecting electrodes 32, 38 can form a circular band that can extend around all or portions of the limb 302, and in some embodiments, one or both of the current injecting electrodes 32, 38 can be planar.

In some embodiments, the current injecting electrodes 32, 38 can include an electrically conductive surface that can conductively contact the limb 302 to inject current into the limb 302. This electrically conductive surface can include a metal or any other material capable of injecting current into the limb 302. In some embodiments, the current injecting electrodes 32, 38 may be MEMS chip-electrodes, and the electrically conductive surface can be one or several arrays of approximately 400-nanometer conductive needles that reduce the impedance at the skin interface without causing discomfort or increasing the risk of infection or bio-fouling of the electrode skin interface.

A variety of features and/or geometries can be used to attach the current injecting electrodes 32, 38 to the limb 302. As discussed above, the current injecting electrodes 32, 38 can form a circular band. The circular band can define a central volume that can receive the limb 302 to facilitate attachment of the current injecting electrodes 32, 38 to the limb. In some embodiments, the current injecting electrodes 32, 38 can include one or several features that allow and/or cause the constriction of the current injection electrodes 32, 38 on or around the limb 302 to thereby secure the current injecting electrodes 32, 38. In some embodiments, such as, for example, when the current injecting electrodes 32, 38 form a circular band, these features that allow and/or cause constriction can include, for example, an elastic band, a drawstring, a snap, a zipper, a button, or a tie. In some embodiments, the current injecting electrodes 32, 38 can include an adhesive that allows the current injecting electrodes 32, 38 to be affixed to a portion of the limb 302 of a patient.

The connection points of the current injecting electrodes 32, 38 can allow for physical or communicating connection of the current injecting electrodes 32, 38 with other components of the potential disease state detection device 10. In some embodiments, the connection points can be configured to receive control signals, instructions, and/or power from other components of the potential disease state detection device 10, and in some embodiments, the connection points can be configured to provide information to the other components of the potential disease state detection device 10. In some embodiments, the connection points can be wires, contacts, leads, or any other features that can allow physical connection of the current injecting electrodes 32, 38 to the other components of the potential disease state detection device 10. In some embodiments, the connection points can be a receiver, a transmitter, an antenna, or any other feature that allows the communicating connection between the current injecting electrode 32, 38 and the other components of the wireless system.

The current sensing electrodes 34, 36 can sense aspects of the current received from the current injecting electrode 32, 38 and transmitted through the limb 302. The sensed aspects can include, for example, the current, voltage, phase, or any other desired current property. As such, the current injecting electrodes 32, 38 can comprise a conductive material configured to receive transmit current from the limb 302, connection points configured to allow connection of the current sensing electrodes 34, 36 to a measurement source, and features configured to attach the current sensing electrodes 34, 36 to the limb 302.

The current sensing electrodes 34, 36 can comprise a variety of shapes and sizes. Like the current injecting electrodes 32, 38, the current sensing electrodes 34, 36 can form a band, such as, for example, a circular band, or the current sensing electrodes 34, 36 can be planar.

Like the current injecting electrodes 32, 38, in some embodiments, the current sensing electrodes 34, 36 can include an electrically conductive surface that can conductively contact the limb 302 to allow sensing of the injected current. This electrically conductive surface can include a metal or any other material capable of injecting current into the limb 302. In some embodiments, the current sensing electrodes 34, 36 can be MEMS chip-electrodes, and the electrically conductive surface can be one or several arrays of approximately 400-nanometer conductive needles that reduce the impedance at the skin interface without causing discomfort or increasing the risk of infection or bio-fouling of the electrode skin interface.

A variety of features and/or geometries can be used to attach the current sensing electrodes 34, 36 to the limb 302, including, for example, mechanical features such as, for example, elastic bands, snaps, straps, buttons, zippers, adhesives, and ties.

The connection points of the current sensing electrodes 34, 36 can allow for physical or communicating connection of the current sensing electrodes 34, 36 with other components of the wireless system 300. In some embodiments, the connection points can be configured to receive control signals, instructions, and/or power from other components of the potential disease state detection device 10, and in some embodiments, the connection points can be configured to provide information to the other components of the potential disease state detection device 10. In some embodiments, the connection points can be wires, contacts, leads, or any other features that can allow physical connection of the current sensing electrodes 34, 36 to the other components of the potential disease state detection device 10. In some embodiments, the connection points can be a receiver, a transmitter, an antenna, or any other feature that allows the communicating connection between the current sensing electrodes 34, 36 and the other components of the wireless system.

In some embodiments, the current injecting electrodes 32, 38 and the current sensing electrodes 34, 36 can be separate components, and in some embodiments, the functionalities of the current injecting electrodes 32, 38 and of the sensing electrodes 34, 36 can be combined. Thus, in such a combined embodiment, an electrode can both inject current and sense current injected by another electrode.

An electrode pair, including, for example, two electrodes 32, 34, 36, 38 including at least one current injecting electrode 32, 38 and at least one current sensing electrode 34, 36 can be placed on any desired portion of a limb 302. In some embodiments, the placement of the electrode pair can allow for the detection of a potential disease state in all or portions of the limb 302. As seen in FIG. 3, the current injecting electrodes 32, 38 and the current sensing electrodes 34, 36 are divided into a first electrode pair including a first current injecting electrode 32 and a first current sensing electrode 34, and a second electrode pair, including a second current injecting electrode 38 and a second current sensing electrode 36. As seen in FIG. 3, the first electrode pair 32, 34 is positioned to allow collection of data from at least the anterior compartment of the limb 302, and the second electrode pair 38, 36 is positioned to allow collection of data from at least the lateral compartment of the limb 302. Positioning of the electrodes will be discussed in greater detail below, however, the specific positioning of the electrodes 32, 34, 36, 38 can affect the data that can be gathered from the limb 302 and can provide the ability to localize detection of a potential disease state.

The potential disease state detection device 10 includes a patch 304. The patch 304 can serve as an intermediate component between the electrodes 32, 34, 36, 38 and the other components of the potential disease state detection device 10. In some embodiments, the patch 304 can be co-located with one or all of the electrodes 32, 34, 36, 38 or integrated into one or all of the electrodes 32, 34, 36, 38. The patch 304 can include power features that can store power, generate power, and/or receive power from the potential disease state detection device 10, information features to send and receive information and/or instructions from other components of the potential disease state detection device 10 and/or to store information, and attachment features to attach the patch 304 to the limb 302.

The power features of the patch 304 can be any features that can store, receive, and/or generate sufficient power to allow generation and gathering of information from the electrodes 32, 34, 36, 38 relating to a potential disease state. In some embodiments, these features can include one or several batteries, one or several capacitors, inductive power transmission features, strain-based power generation features, or any other similar feature.

The information features of the patch 304 can be any features that can control the electrodes 32, 34, 36, 38 and collect and analyze data generated by the electrodes 32, 34, 36, 38. The information features can be configured to send and receive information from the electrodes and to send and receive information from the other components of the potential disease state detection device 10. In some embodiments, the information features can include a memory that stores information received from the electrodes 32, 34, 36, 38 and transmission and receiving features that transmit information to the electrodes 32, 34, 36, 38 and/or to the other components of the potential disease state detection device 10.

The attachment features of the patch 304 can be similar to those discussed above in relation to the electrodes 32, 34, 36, 38.

The potential disease state detection device 10 can include a controller 20 or base unit. The controller 20 can provide power and/or instructions to the patch 304, receive information from the patch 304, and analyze the received information. The controller 20 is discussed in greater detail above.

The controller 20 can be configured to control the current injection and processing of data received from the patch 304. In some embodiments, the controller 20 can control, for example, testing parameters such as the frequency of sampling, the parameters of the injected current (i.e. voltage, current, frequency). In one embodiment, for example, data can be gathered at intervals specified by a user, which intervals can be regular and/or irregular. In some embodiments, the sampling rate can be up to 5 Hz, up to 10 Hz, up to 20 Hz, up to 60 Hz, up to 100 Hz, or any other or intermediate sampling rate.

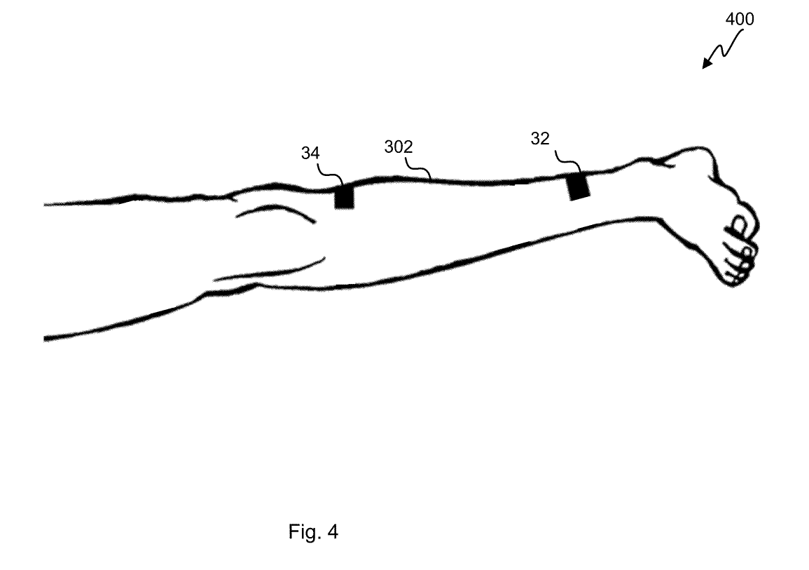

The electrodes 32, 34, 36, 38 can be placed in any desired position on the limb 302. The placement of the electrodes 32, 34, 36, 38 on the limb can affect the data that the electrodes 32, 34, 36, 38 generate and can affect the ability of the potential disease state detection device 10 to identify the potential disease state. The electrodes 32, 34, 36, 38 can be radially and/or longitudinally place on the limb 302. FIGS. 4 through 6 depict different embodiments of placements of electrodes 32, 34, 36, 38 on the limb 302. The details of the placement of the electrodes 32, 34, 36, 38 on the limb 302, and effects of such placements will be discussed below.

FIG. 4 depicts a placement 400 of the electrodes 32, 34 around the limb 302. Specifically, FIG. 4 depicts the placement 400 of an electrode pair having a current injecting electrode 32 placed on the distal, posterior portion of the limb 302, and a current sensing electrode 34 placed on a relatively more proximal, posterior portion of the limb 302. As discussed above, the electrodes 32, 34, 36, 38 can be shaped so as to encircle all or portions of the limb 302 or can be planar so as to allow placement on a surface of the limb 302. In the placement 400 depicted in FIG. 4, electrodes 32, 34 are planar and are placed on the posterior portion of the limb 302. In embodiments in which the electrodes 32, 34 are shaped so as to encircle all or portions of the limb 302, the electrodes 32, 34 may radially wrap around the limb 302. Advantageously, the placement of one or both of the electrodes 32, 34 over a radial portion of the limb 302 can allow the detection of the potential disease state in a portion of the limb 302, such as, for example, within a single muscle compartment. Referring again to FIG. 4, the specific placement of electrodes 32, 34 can allow the detection of the potential disease state in the posterior portion of the limb 302.

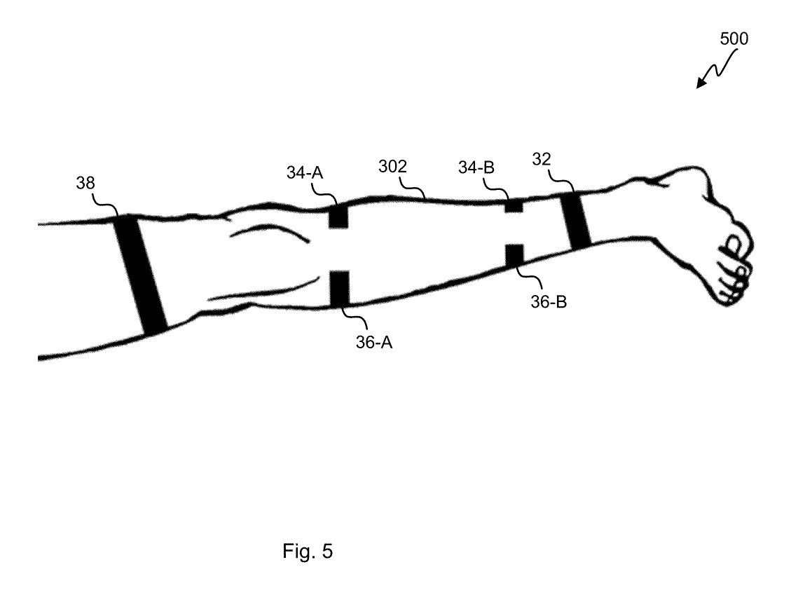

FIG. 5 depicts a placement 500 of electrodes 32, 34, 36, 38 on the limb 302. Specifically, FIG. 5 depicts the placement of current injection electrodes 32, 38 respectively placed on the distal and relatively more proximal portions of the limb 302. As seen in FIG. 5, the current injecting electrodes 32, 38 radially encircle the limb 302. FIG. 5 additionally depicts the placement of current sensing electrodes 34-A, 34-B on the posterior portion of the limb 302 and current sensing electrodes 36-A, 36-B on the anterior portion of the limb 302. By placing the current sensing electrodes 34, 36 on different portions of the limb 302, namely, on the posterior and anterior portions of the limb 302 respectively, the current sensing electrodes 34, 36 collect data relating to different regions of the limb 302 and different tissues of the limb 302. Additionally, placement of the current sensing electrodes 34-A, 34-B and current sensing electrodes 36-A, 36-B at different longitudinal positions along the limb 302, the current sensing electrodes 34-A, 34-B and current sensing electrodes 36-A, 36-B further collect data relating to different longitudinal regions of the limb 302 and different tissues of the limb 302. Thus, when current is injected from one or both of current injecting electrodes 32, 38, current sensing electrodes 34 can generate data relating to the potential disease state of posterior tissues of the limb 302 and current sensing electrodes 36 can generate data relating to the potential disease state of anterior tissues of the limb 302. Similarly, when current is injected from one or both of the current injecting electrodes 32, 38 the current sensing electrodes 34-A, 34-B and current sensing electrodes 36-A, 36-B can generate data relating to the potential disease state of tissues at different longitudinal positions along the limb 302.

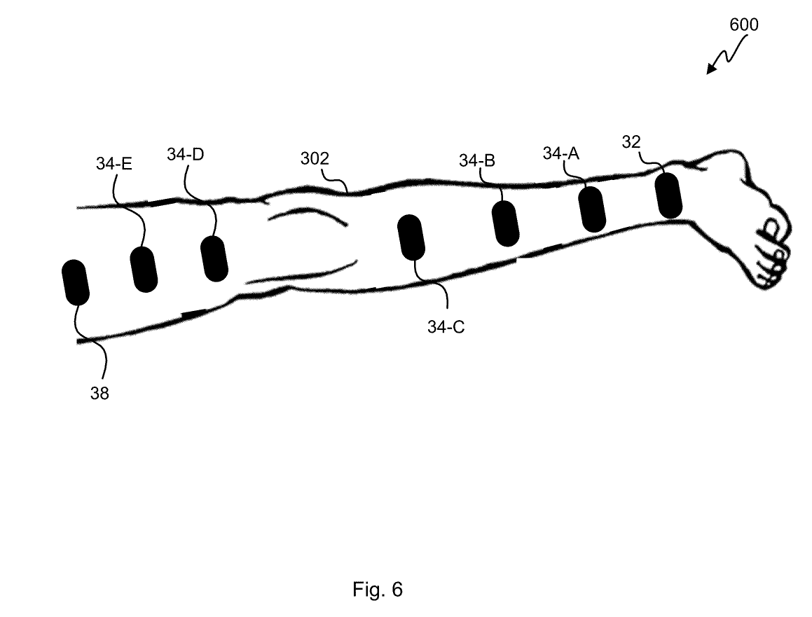

FIG. 6 depicts a placement 600 of electrodes 32, 34, 38 on the limb 302. Specifically, FIG. 6 depicts the placement of current injection electrodes 32, 38 respectively placed on the distal and relatively more proximal portions of the limb 302, and the longitudinal placement of electrodes 34-A, 34-B, 34-C, 34-D, 34-E progressing from the distal end to relatively more proximal positions respectively. Additionally, placement of the current sensing electrodes 34-A, 34-B, 34-C, 34-D, 34-E at different longitudinal positions along the limb 302, allows collection of data relating to different regions of the limb 302 and different tissues of the limb 302. Thus, when current is injected from one or both of the current injecting electrodes 32, 38 the current sensing electrodes 34-A, 34-B, 34-C, 34-D, 34-E can generate data relating to the potential disease state of tissues at different longitudinal positions along the limb 302. This can advantageously allow the localized detection of a potential disease state and targeted treatment of the diseased tissue.

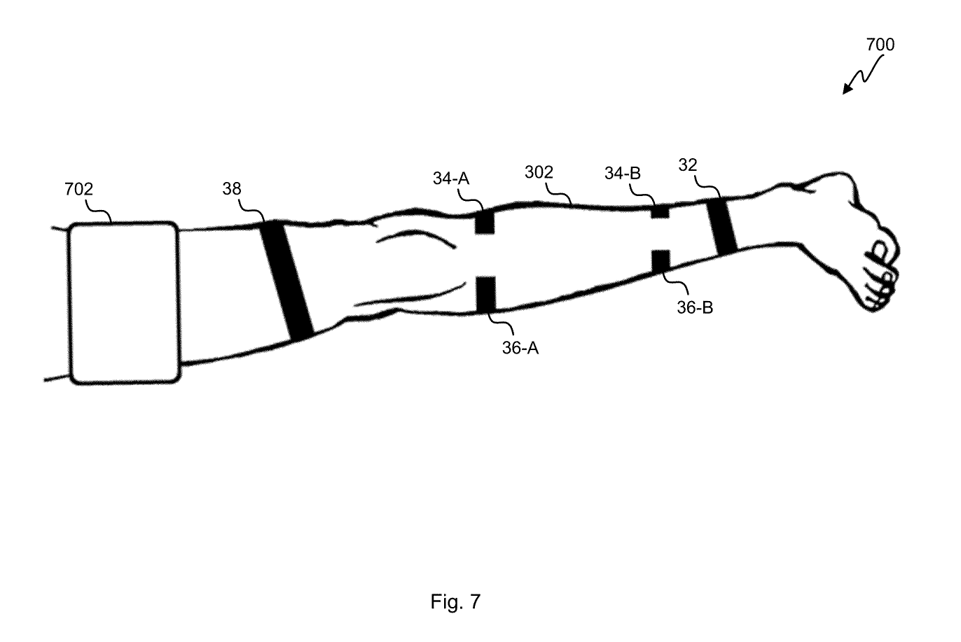

FIG. 7 depicts a placement 700 of electrodes 32, 34, 36, 38 and a cuff 702 on the limb 302. Specifically, FIG. 7 depicts the same electrode 32, 34, 36, 38 placement depicted in FIG. 5, but additionally depicts the placement of the cuff 702 at the proximal portion of, and encircling, the limb 302. The cuff 702 can be any object capable of selectively applying a force to the limb 302 to thereby restrict flow and/or circulation to and/or from portions of the limb 302 relatively more distal than the cuff 702, and can be, for example, a tourniquet or a blood pressure cuff. Advantageously, the relatively more proximal placement of the cuff 702 on the limb 302 shown in FIG. 7 allows use of the cuff to apply force to the limb 302 to thereby restrict flow and/or circulation to and/or from the portions of the limb 302 having the attached electrodes 32, 34, 36, 38.

In some embodiments having the placement 700 depicted in FIG. 7, the cuff 702 may be used to perform a stress test on the limb 302, and specifically, the cuff 702 can apply a force to the limb 302 to thereby restrict flow and/or circulation to and/or from the limb, resulting in the accumulation of fluid in the limb 302. Data relating to the accumulation of fluid in the limb 302 can be collected by the electrodes 32, 34, 36, 38. After a desired time, the cuff 702 can be released and flow and/or circulation from the limb 302 can be restored, which restored flow can result in the depletion of the accumulated fluid in the limb 302. The electrodes 32, 34, 36, 38 can collect data relating to the rate of the depletion of the accumulated fluid in the limb 302, which data can be used to detect a potential disease state in the limb 302 or in specific portions of the limb (i.e., anterior or posterior portions or relatively more distal or more proximal portions).

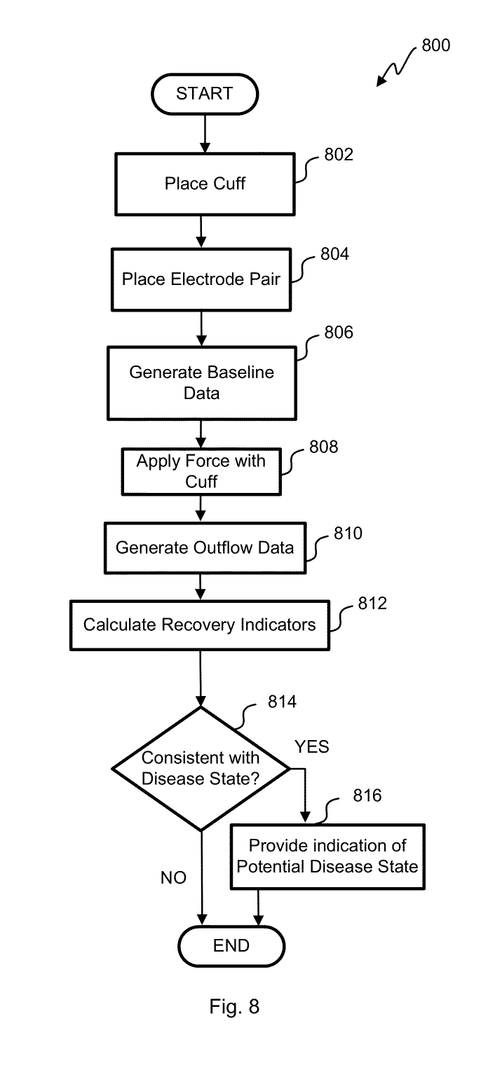

FIG. 8 is a flowchart depicting one embodiment of a process 800 for performing a stress test with the cuff 702 to detect a potential disease state in a limb 302. The process 800 begins at block 802, wherein the cuff 702 is placed on the limb 302. In some embodiments, the cuff 702 can be placed relatively proximal to the areas of the limb 302 on which the stress test will be performed. In some embodiments, the cuff 702 is placed in the most proximal position on the limb 302 possible.

After the cuff 702 is placed, the process 800 proceeds to block 804, wherein the electrode pair is placed on the limb 302. The electrode pair can comprise two or more electrodes 32, 34, 36, 38, which can be placed on the limb 302 to allow detection of the potential disease state in desired portions of the limb 302.

After the electrode pair is placed, the process 800 proceeds to block 806, wherein baseline data is generated. In some embodiments, the generation of baseline data can include the energizing of the electrode pair and the collection of data with the electrode pair. The generation of baseline data can be performed over a sufficient period of time such that the baseline data represents a steady-state condition of the limb 302.

After the baseline data is generated, the process 800 proceeds to block 808, wherein a force is applied to the limb 302 with the cuff 702. In some embodiments, the application of force to the limb 302 can be achieved by the tightening of a tourniquet or the inflation of a blood pressure cuff. In some embodiments, a force is applied to the limb 302 with the cuff 702 so as to generate a pressure on the limb 302 larger than the diastolic blood pressure, larger than the systolic blood pressure, or larger than the diastolic blood pressure and lower than the systolic blood pressure. Advantageously, the generation of pressure on the limb 302 larger than the diastolic blood pressure prevents the outflow of blood from the limb, generation of pressure on the limb 302 larger than the systolic blood pressure prevents the inflow of blood to, and the outflow of blood from the limb 302, and the generation of pressure larger than the diastolic blood pressure and smaller than the systolic blood pressure allows the inflow of blood to the limb while preventing the outflow of blood from the limb. To facilitate the application of the correct amount of force to the limb 302, in some embodiments, the blood pressure of the individual undergoing the stress test can be measured before the start of the test. The size of the cuff can also be varied and/or selected to selectively obstruct desired vessels, such as either the superficial veins, deep veins, or both superficial and deep veins. Wider cuffs are often used to obstruct all veins, while narrow cuffs can be used to only close off veins to a certain depth in the leg.

The force can be applied to the limb 302 with the cuff 702 for a desired amount of time. In some embodiments, the force can be applied for a long enough time to allow the accumulation of sufficient fluid so as to require at least 5 seconds, 10 seconds, 20 seconds, 50 seconds, 100 seconds, 200 seconds, 300 seconds, 500 seconds, 1,000 seconds, or any other or intermediate amount of time that passes before the limb 302 returns to its baseline volume or achieves a steady state condition after the force applied by the cuff 702 is removed.

After the desired force is applied to the limb 302 by the cuff 702 for the desired amount of time, the process 800 moves to block 810 wherein outflow data is generated. In some embodiments, the generation of outflow data can include the energizing of the electrode pair and the collection of data with the electrode pair. The generation of outflow data can be performed over a sufficient period of time to allow the limb 302 to return to the steady-state condition represented by the baseline data.

After the outflow data is generated, the process 800 proceeds to block 812, wherein recovery indicators are calculated. The recovery indicators can be an indication of the speed with which the stress tested limb 302 returns to the steady-state condition represented by the baseline data. In some embodiments, the recovery indicators can include an expelled volume and standardized time constant (τ). The expelled volume and the standardized time constant (τ) can be calculated according to the following equations.

$$\begin{matrix}{Volume}_{Expelled} = \frac{{Volume}_{Max} - {Volume}_{Min}}{{Volume}_{Max}} \times 100 & ( 1 ) \\ {Time}_{Recovery} = time ( τ * {Volume}_{original} ) & ( 2 )\end{matrix}$$

After the recovery indicators are calculated, the process 800 proceeds to decision state 812, wherein it is determined if the recovery indicators are consistent with the potential disease state. This determination can be made by comparing the recovery indicators to threshold values. In the case of expelled volume and the above calculated standardized time constant (τ), a relatively lower expelled volume and a smaller standardized time constant (τ) are indicators of a relatively larger potential disease state. If the recovery indicators are consistent with a potential disease state, then the process 800 proceeds to block 816 wherein an indication of the potential disease state is provided. After the indication of the potential disease state is provided, or if the recovery indicators are inconsistent with a potential disease state, the process can terminate.

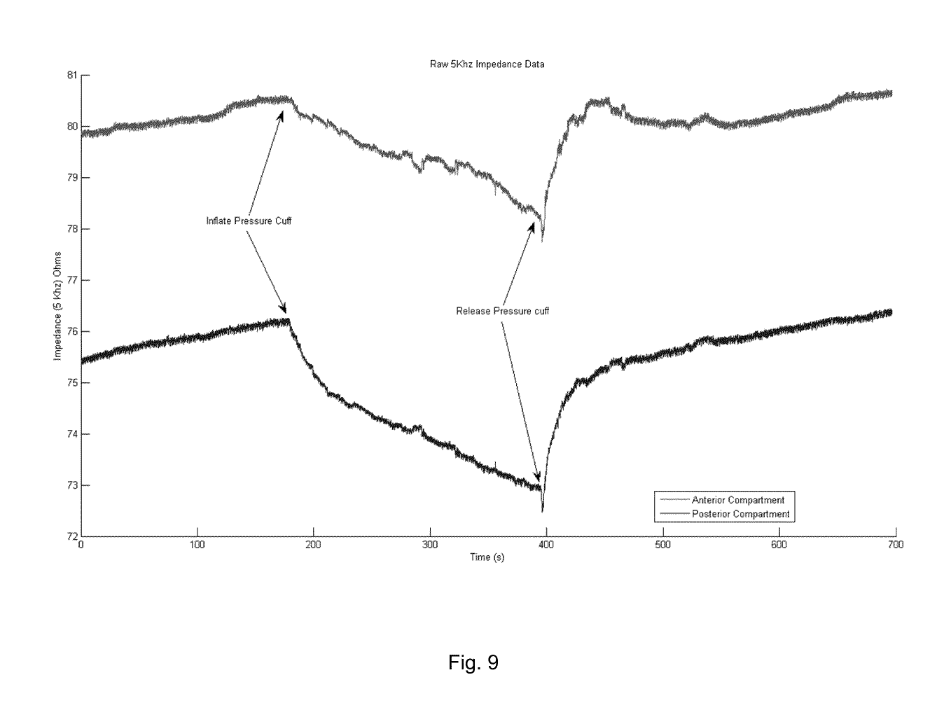

FIGS. 9 through 11B are graphs illustrating data collected during a stress test performed with the cuff 702 and electrodes 32, 34, 36, 38. Specifically, FIG. 9 is a graph illustrating a patient's measured impedance (Ohms) versus time. The data shown in FIG. 9 was calculated using the 5 Khz current injection frequency. The top data line shown in FIG. 9 indicates impedance recorded by anteriorly positioned electrodes 32, 34, 36, 38 from an anterior muscle compartment and the bottom line shown in FIG. 9 indicates impedance recorded by posteriorly positioned electrodes 32, 34, 36, 38 from a posterior muscle compartment. As indicated in FIG. 9, the measured impedance data changes when the cuff 702 was inflated and when the cuff 702 was deflated.

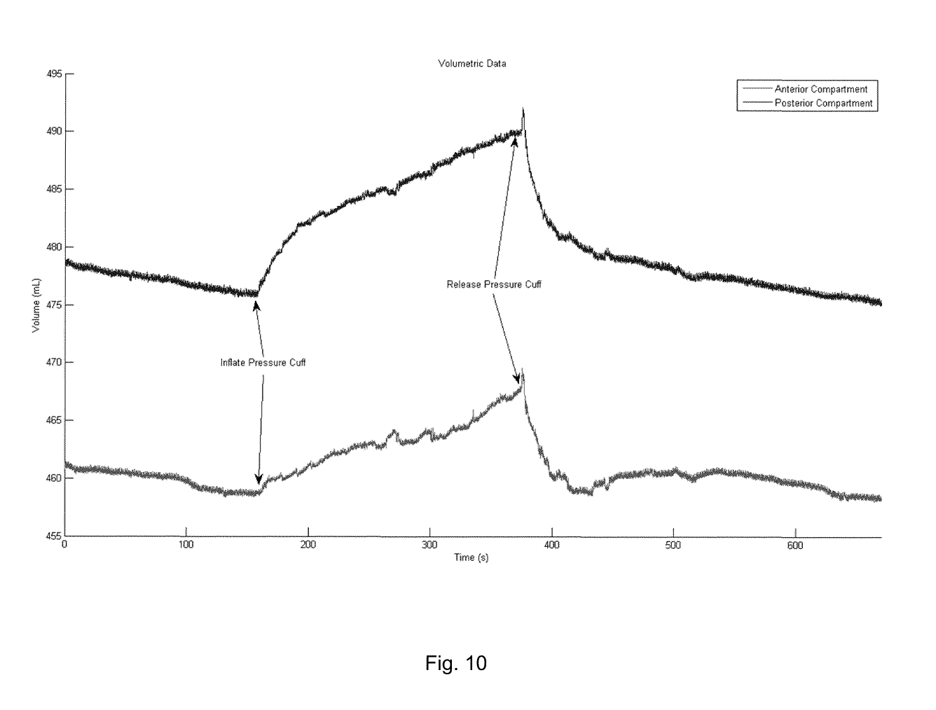

FIG. 10 is a graph illustrating volume (mL) of an anterior muscle compartment (top data line) and of a posterior muscle compartment (bottom data line) versus time. The volume of a limb 302 and/or of muscle compartments of that limb 302 can be calculated from collected impedance using a variety of techniques. The volume data depicted in FIG. 10 was calculated form the impedance data depicted in FIG. 9 using the Cole-Cole model. As indicated in FIG. 10, the calculated volume data begins to significantly change when the cuff 702 was inflated and when the cuff 702 was deflated.

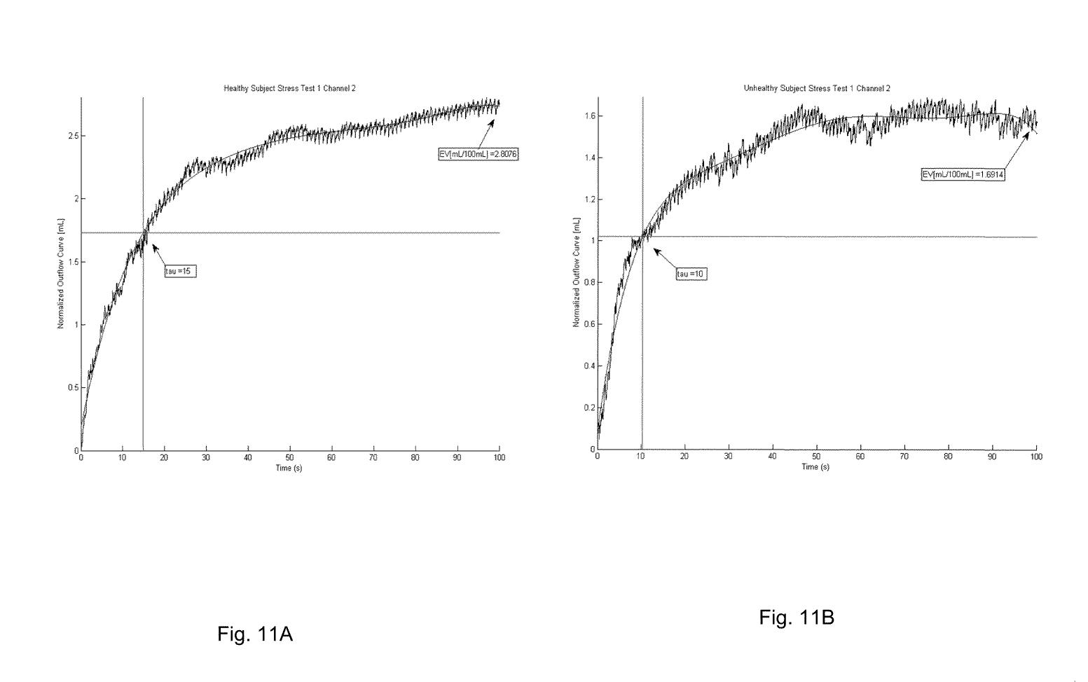

FIGS. 11A and 11B are graphs showing the volume release curves of a healthy patient and of an unhealthy patient. The volume release curves plot nominal outflow (mL) versus time (s). The volume release curves also indicate the standardized time constant (τ) and the expelled volume (EV). As indicated above, the healthy patient has both a larger expelled volume and a larger standardized time constant (τ) than the unhealthy patient. Accordingly, the potential disease state of both patients is indicated by the standardized time constant (τ) and the expelled volume, with these values indicating the healthy condition of the healthy patient and the diseased condition of the unhealthy patient.

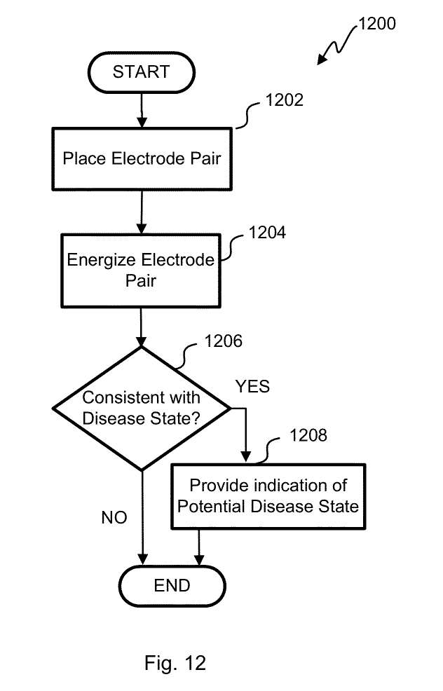

FIG. 12 is a flowchart depicting one embodiment of a process 1200 for detecting a potential disease state in a limb 302. The process 1200 can be used in the detecting of a potential disease state in a limb 302 by determining whether a signal generated by an electrode pair is consistent with the potential disease state. In some embodiments, process 1200 can be used, for example, in detection of compartment syndrome, including acute compartment syndrome by monitoring the pressure of the limb and/or muscle compartments where compartment syndrome is suspected.

The process 1200 begins at block 1202, wherein the electrode pair is placed on the limb 302. The electrode pair can comprise two or more electrodes 32, 34, 36, 38, which can be placed on the limb 302 to allow detection of the potential disease state in desired portions of the limb 302.

After the electrode pair is placed on the limb 302, the process 1200 proceeds to block 1204, wherein the electrode pair is energized. In some embodiments, the bioimpedance controller 20 and/or the controller 52 can energize the electrode pair by sending power to the current injecting electrode 32, 38 of the electrode pair, or by sending an instruction to the patch 304 to power the current injecting electrode 32, 38 of the electrode pair. As discussed above, the electrode pair can be energized with current having the desired frequency, current, and voltage properties, which properties can be selected in accordance with the desired testing. Thus, as discussed above, a different frequency may be used if the ECF volume is being measured than if the ICF volume is being measured.

In one specific embodiment, the electrode pair can be energized when the controller 52 directs the DDS 56 to generate a current signal having a desired frequency, magnitude, and phase. The current signal can be applied to the bandpass filter 58, which passes the desired band of frequencies to the differential current source 60, at the frequency set by the DDS 56. This current can then be injected into the limb of the patient by current injecting electrode 32, 38.

As the electrode pair is energized, the current sensing electrode 34, 36 can begin to collect current data which can include, for example, current, phase, or voltage information. This current data can relate to the tissue of the limb 302 that is completing the circuit in the electrode pair. In one embodiment, after data signals from the current sensing electrode 34, 36 can be stored on the memory chip or card for a period of time (e.g., for 24 hours or more) so that the data can be uploaded via the wireless communication on demand when desired. The data signals can be applied to a differential amplifier 70, which can produce a signal that can be input to a bandpass filter 72. The resulting filtered signal can be input to an analog-digital converter (ADC) 74, which can convert the analog voltage level to a corresponding digital signal that can be input to controller 52 for processing.

After the electrode pair is energized, the process 1200 proceeds to decision state 1206, wherein it is determined if the current data is consistent with the potential disease state. In some embodiments, this determination can be made by the bioimpedance controller 20 and/or the controller 52. If the current data is consistent with a potential disease state, then the process 1200 proceeds to block 1208 wherein an indication of the potential disease state is provided. The indication of the potential disease state can include, for example, a perceptible signal such as a visible, audible, or tactile signal. After the indication of the potential disease state is provided, or if the recovery indicators are inconsistent with a potential disease state, the process 1200 can terminate.

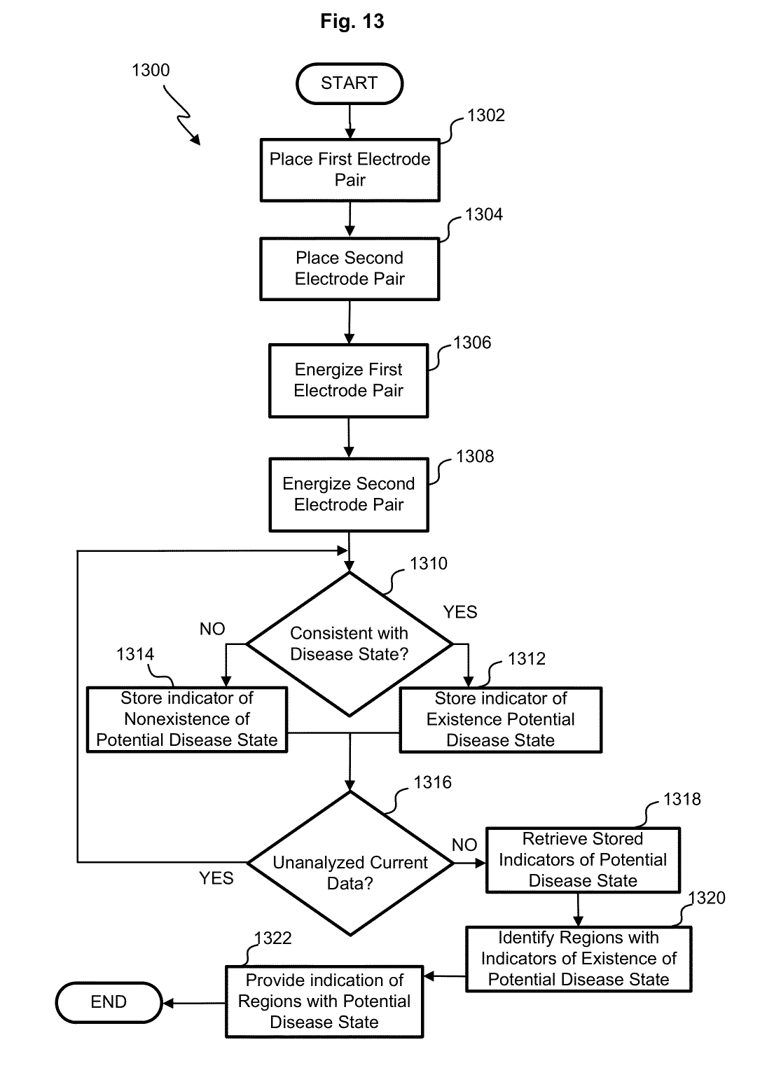

FIG. 13 is a flowchart depicting one embodiment of a process 1300 for detecting a potential disease state in a region of the limb 302. The process 1300 can be used in the detecting of a potential disease state in a limb by determining whether a signal generated by an electrode pair is consistent with the potential disease state. In some embodiments, process 1300 can be used, for example, to localize a potential disease state in a limb, or in portions of a limb. Specifically, in some embodiments, process 1300 can be used in assisting in treating unresponsive patients, and in the detection of compartment syndrome, including acute compartment syndrome by monitoring the pressure and/or volume of the limb and/or muscle compartments where compartment syndrome is suspected. Advantageously, process 1300 can be used to localize the compartment syndrome to identify the potentially affected compartment. This identification of the potentially affected compartment can assist in surgical planning by providing a physician with information regarding which compartments may potentially affected by compartment syndrome, and which compartments may require a fasciotomy.

The process 1300 begins at block 1302, wherein a first electrode pair is placed on a first region of the limb 302. The first electrode pair can comprise two or more electrodes 32, 34, 36, 38, which can be placed on the limb 302 to allow detection of the potential disease state in the first region of the limb 302.

After the first electrode pair is placed on the first region of the limb 302, the process 1300 proceeds to block 1304, wherein a second electrode pair is placed on a second region of the limb 302. The second electrode pair can comprise two or more electrodes 32, 34, 36, 38, which can be placed on the limb 302 to allow detection of the potential disease state in the second region of the limb 302.

After the second electrode pair is placed on the limb 302, the process 1300 proceeds to block 1306, wherein the first electrode pair is energized. The first electrode pair can be energized when the current injecting electrode 32, 38 receives the current signal from, or at the direction of the bioimpedance controller 20 and/or the controller 52. As discussed above, in some embodiments, the energizing of the first electrode pair can additionally include the collection, storage, and/or processing of first current data by the current sensing electrode 34, 36 and/or other component of the potential disease state detection device 10.

After the first electrode pair is energized, the process 1300 proceeds to block 1308, wherein the second electrode pair is energized. The second electrode pair can be energized when the current injecting electrode 32, 38 receives the current signal from, or at the direction of the bioimpedance controller 20 and/or the controller 52. As discussed above, in some embodiments, the energizing of the second electrode pair can additionally include the collection, storage, and/or processing of second current data by the current sensing electrode 34, 36 and/or other components of the potential disease state detection device 10.

After the second electrode pair is energized, the process 1300 proceeds to decision state 1310, wherein it is determined if the current data is consistent with the potential disease state. In some embodiments, this determination can be made by the bioimpedance controller 20 and/or the controller 52. In embodiments including multiple electrode pairs, this determination may be made on current data received from one or all of the electrode pairs.

If the current data is consistent with a potential disease state, then the process 1300 proceeds to block 1312 wherein an indicator of the existence of the potential disease state is stored. In some embodiments, the indicator of the existence of the potential disease state can include information relating to the current data, such as, for example, time it was collected, the identification of the electrode pair from which it was collected, the identification of the region of the limb 302 from which it was collected, or any physical data associated with the patient from which the current data was collected.

If the current data is not consistent with a potential disease state, then the process 1300 proceeds to block 1314 wherein an indicator of the nonexistence of the potential disease state is stored. In some embodiments, the indicator of the nonexistence of the potential disease state can include information relating to the current data, such as, for example, the time or conditions in which it was collected, the identification of the electrode pair from which it was collected, the identification of the region of the limb 302 from which it was collected, or any physical data associated with the patient from which the current data was collected.

The indicator of the existence of the potential disease state and/or the indicator of the nonexistence of the potential disease state can be stored within the potential disease state detection device 10, including, for example, the storage such as the memory chip or card.

After the indicator of either the existence of the potential disease state or the nonexistence of the potential disease state is stored, the process 1300 proceeds to decision state 1316, wherein it is determined whether there is any unanalyzed current data from one of the electrode pairs. As mentioned above, in some embodiments, some or all of the current data received from the electrode pairs may be analyzed at decision state 1310. If it is determined that there is unanalyzed current data, then the process 1300 returns to decision state 1310 wherein the unanalyzed data is analyzed.

If it is determined that there is no unanalyzed current data, then the process 1300 proceeds to block 1318, wherein the stored indicators of the potential disease state are retrieved. In some embodiments, the indicators are retrieved from part of the potential disease state detection device 10 such as, for example, the storage such as the memory chip or card.

After the stored indicators of the potential disease state are retrieved, the process 1300 proceeds to block 1320, wherein regions of the limb 302 associated with indicators of the existence of the potential disease state are identified. In some embodiments, this identification can correspond to identifying the electrode pair from which the current data was collected and/or identifying the region of the limb to which the electrode pair was attached or with which the generated current data is otherwise associated.

After the regions associated with indicators of the existence of the potential disease state are identified, the process 1300 proceeds to block 1322, wherein an indication of regions associated with the potential disease state is provided. In some embodiments, the indication of the regions of the limb 302 associated with the potential disease state can include, for example, a perceptible signal such as, a visible, audible, or tactile signal. In some embodiments, the indication of the regions of the limb 302 associated with the potential disease state can identify the electrode pair that generated the current data that is consistent with the potential disease state, the region of the limb 302 on which the electrode pair is attached that generated the current data that is consistent with the potential disease state or with which the current data that is consistent with the disease state is associated. In some embodiments, each region associated with current data that is consistent with the potential disease state can be identified. In some embodiments, the indication of the regions of the limb 302 associated with the potential disease state can include, for example, a potential recommended treatment, such as, potential fasciotomies for muscle compartments with elevated pressures. After the indication of regions associated with the potential disease state is provided, the process 1300 terminates.

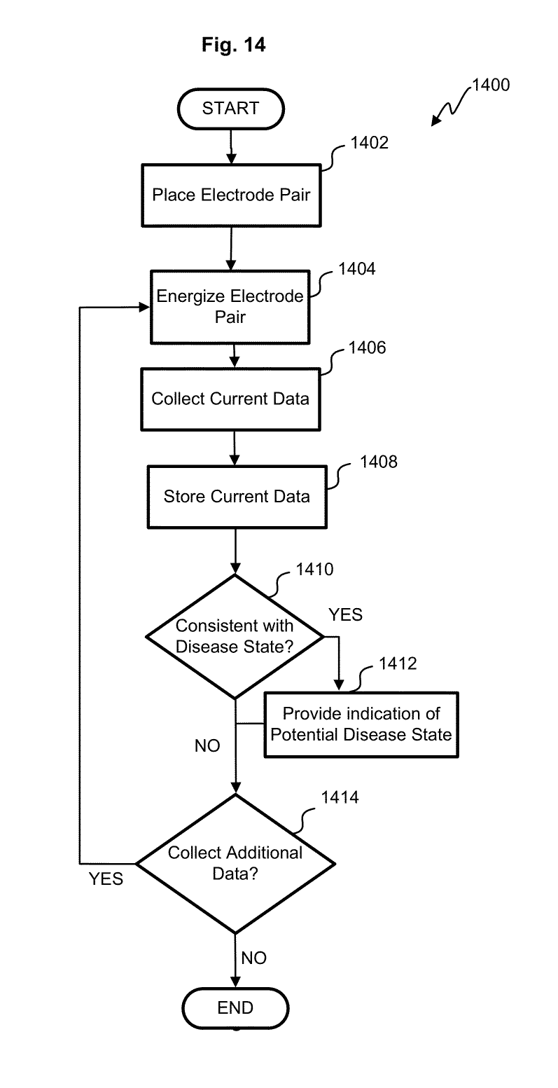

FIG. 14 is a flowchart depicting one embodiment of a process 1400 for identifying a potential disease state of a limb 302 of a patient over time. The process 1400 can be used in the detecting of a potential disease state in the limb 302 by tracking limb information over a period of time. This information can be used to see whether the limb 302 is maintaining its current state, or if its state is improving or deteriorating. In some embodiments, the process 1400 can be additionally used to trigger modifications in a treatment or to trigger a treatment.

The process 1400 can be used, for example, to perform an ambulatory plethysmography wherein the patient can be monitored during the performing of routine tasks, or tasks representative of routine tasks. The data gathered from this testing can be used to categorize activities as beneficial, neutral, or deleterious to the patient's condition.

The process 1400 can be used, for example, to perform monitoring such as venous monitoring to monitor a patient's vascular health, venous insufficiency monitoring to indicate potential venous valvular insufficiency and to provide insight into tasks and activities that contribute to or are affected by the insufficiency, thrombus monitoring, including monitoring patients who are at risk of developing a venous thrombus for signs of a potential thrombus forming or breaking loose, monitoring the treatments of a patient after they have been diagnosed as thrombotic, and/or monitoring patients during relatively long periods of inactivity for signs of decreased circulation and/or increased risk of thrombus formation, medication plan/efficacy monitoring to determine whether a medication plan is having a desired effect on a patient's circulation and/or having a deleterious effect on a patient's circulation, or exercise monitoring, including monitoring the vascular status of athletes undergoing high-performance tests to assist in quantification of incremental changes to a training regimen and to provide feedback to an athlete and a coach, or monitoring for a circulatory ailment such as chronic exertional compartment syndrome, and to determine the severity and causes of the ailment.

In each of the above mentioned cases, the process 1400 is part of the development of a response or treatment to the gathered data. Thus, in the case of venous monitoring, gathered values indicating venous problems could be used in creating a treatment plan. In the case of thrombus monitoring gathered values indicating a risk of thrombus could be used to trigger an alarm, to request care, to prompt a patient to increase his activity level to increase circulation, or to control a device configured to stimulate circulation in the patient's limb or limbs, such as by, for example, constricting the limb or limbs. In the case of the application of the process 1400 to medication plan/efficacy monitoring, the gathered values can be used to adjust dosages or to change a patient's drugs based on the impact of the current medication plan on the patient, and in the case of exercise monitoring, gathered values can be used to create or improve a training plan or to create a treatment plan responsive to a detected vascular ailment.

The process 1400 can be used in detecting potential circulatory problems such as acute compartment syndrome, in treating unresponsive patients, in pre-hospital triage situations, in patients with trauma including extremity and polytrauma, and in surgical monitoring or surgical planning by generating values corresponding to pressures and volumes of a limb or muscle compartments in a limb, and/or tracking changes in the pressures and volumes of a limb or muscle compartments in a limb. Further, in each of the above mentioned cases, the process 1400 is part of the development of a response or treatment to the gathered data. Thus, in the case of the compartment syndrome, unresponsive patients, pre-hospital triage situations, patients with trauma including extremity and polytrauma, and in surgical monitoring, the process 1400 can include providing an indication of potentially appropriate treatments based on the gathered values. These treatments can include, performing a fasciotomy, performing a venous ligation or repair, performing an arterial ligation or repair, performing venous ablation, performing therapy, or any other similar treatment.

The process 1400 begins at block 1402, wherein an electrode pair is placed on a region of the limb 302. The electrode pair can comprise two or more electrodes 32, 34, 36, 38, which can be placed on the limb 302 to allow detection of the potential disease state in the region of the limb 302.

After the electrode pair is placed on the region of the limb 302, the process 1400 proceeds to block 1404, wherein the electrode pair is energized. The electrode pair can be energized when the current injecting electrode 32, 38 receives the current signal from, or at the direction of the bioimpedance controller 20 and/or the controller 52.

After the electrode pair is energized, the process 1400 proceeds to block 1406, wherein the current data is collected. The current data can include, for example, data relating to the current such as, for example, the voltage detected at the current sensing electrode 34, 36, the current, the phase, or any other dimension related to the current. In some embodiments, current sensing electrodes 34, 36 can collect the current data.

After the current data has been collected, the process 1400 proceeds to block 1408, wherein the current data is stored. In some embodiments, the current data can be stored at or on some component of the potential disease state detection device 10 including, for example, the storage including the memory card or chip. In some embodiments, the storage of the current data can additionally include the processing of current data by the current sensing electrode 34, 36 or other component of the potential disease state detection device 10.

After the current data is stored, the process 1400 proceeds to decision state 1410, wherein it is determined if the current data is consistent with the potential disease state. In some embodiments, this determination can be made by the bioimpedance controller 20 and/or the controller 52.

If the current data is consistent with a potential disease state, then the process 1400 proceeds to block 1412 wherein an indication of the potential disease state is provided. The indication of the potential disease state can be, for example, a perceptible signal such as, for example, a visible, audible, or tactile signal. In some embodiments, the indicator of the existence of the potential disease state can include information relating to the current data, such as, for example, time it was collected, the identification of the electrode pair from which it was collected, or any physical data associated with the patient from which the current data was collected. In some embodiments, the data included in the indicator can facilitate identification of times of day, activities, or conditions that can cause or increase current data indicative of the potential disease state.

In some embodiments, providing an indication of the potential disease state can include, for example, providing a potential treatment plan, providing a potential change in a medication plan, providing an alarm, or triggering a treatment device. In some embodiments, the recommended treatment plan can include, identifying potential surgical treatments, prompting a patient to increase his activity level, requesting medical assistance, identifying potential beneficial therapy, or any other beneficial treatment. In some embodiments, providing a potential change in a medication plan can include providing a potential recommendation to change a dosage of a medication, add a medication, remove a medication, or change a medication. In some embodiments, triggering a treatment device can include sending control signals to a treatment device to, for example stimulate circulation within a patient's extremities by, for example, stimulating muscle contractions within the patient or constricting, either once or repeatedly, at least one of the patient's limbs 302.

After the indication of the potential disease state is provided, or if the current data is not consistent with the potential disease state, the process 1400 proceeds to decision state 1414, wherein it is determined whether to collect additional data. In some embodiments, the collection of data covering a designated time frame can advantageously facilitate in the detection of the potential disease state. This detection of the potential disease state can be based on trends relating to the potential disease state that are apparent over time, such as, for example, the strengthening and/or weakening of data indicative of the potential disease state. Thus, in some embodiments in which a patient has experienced trauma, one or several electrode pairs may be placed on the patient's limbs 302 and may begin collecting and analyzing current data over time to detect developing potential disease states during the patient's healing process. Similarly, in some embodiments, causes or detrimental impacts to the potential disease state of the patient can be investigated by generating and analyzing current data over an extended period of time in which a patient does different things such as, for example, walking, eating, sitting, sleeping, exercising, standing, or laying down. In some embodiments, these different activities can correspond to increased circulation through one or more of the patient's limbs 302 and/or to decreased circulation and/or blood pooling in one or more of the patient's limbs 302 and/or muscles within the limbs 302. If it is determined that additional data is to be collected, then the process 1400 proceeds to block 1404 and energizes the electrode pair. If it is determined that no additional data is to be collected, then the process 1400 terminates.

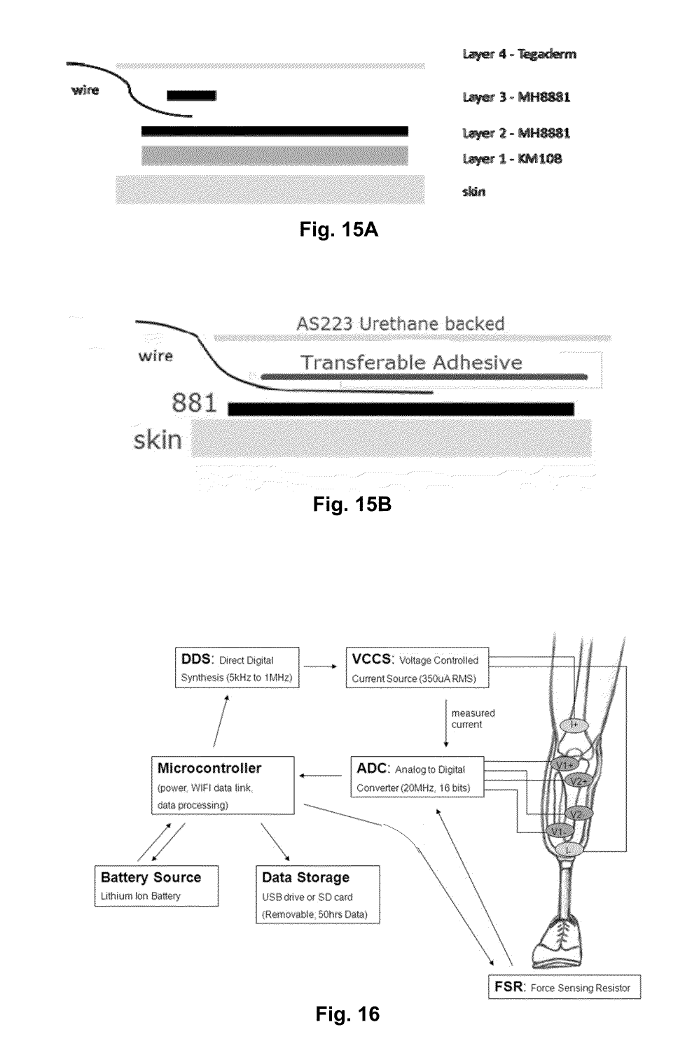

Referring now to FIGS. 15A and 15B, alternative electrode structures are schematically illustrated in cross-section, and may provide a lower resistance than standard, commercially available electrophysiology leads used for heart monitoring and the like. In the embodiment of FIG. 15A, the electrodes comprise three different materials in four discrete layers. Shown at the bottom of the electrode and in direct contact with the skin is a first hydrogel layer (Layer 1), with the exemplary embodiment comprising a thickness in the range of 15 to 32 mil (or 0.015″ to 0.032″) as is available from Katecho, Inc. of Iowa under the designation KM10B hydrogel. Describing the materials in their order of distance away from the skin, the second layer (Layer 2) comprises an adhesive material such as that available from Adhesives Research under the designation ARcare 8881 conductive tape (aka MH8881). This second layer may optionally be cut to the same dimensions as Layer 1. An exposed conductor at the end of the lead wire may be pressed onto the top side of Layer 2, and the exemplary wire may be, for example, an insulated wire such as an 32 AWG (7/40 SPOF with a 0.003″ reinforcing aramid fiber) as may be commercially available from a variety of sources, including New England Wire Tech of Lisbon, N.H. The next material layer (Layer 3) is a smaller strip of MH8881 that covers the exposed conductor. The fourth and final or outermost layer is bandage material such as a piece of Tegaderm™ clear wound bandage material as is commercially available from 3M. Layer 4 is often longer and wider than the underlying layers and thus contacts the skin directly at and/or beyond the peripheral border of the electrode. Referring to FIG. 15B, an alternative electrode design is similar to that described above. Two materials are exchanged: a transferable (very sticky) adhesive is used instead of the small piece of MH8881 above the wire and the outer Tegaderm™ bandage material is replaced with an alternative polymer sheet material such as an AS223 urethane backing.

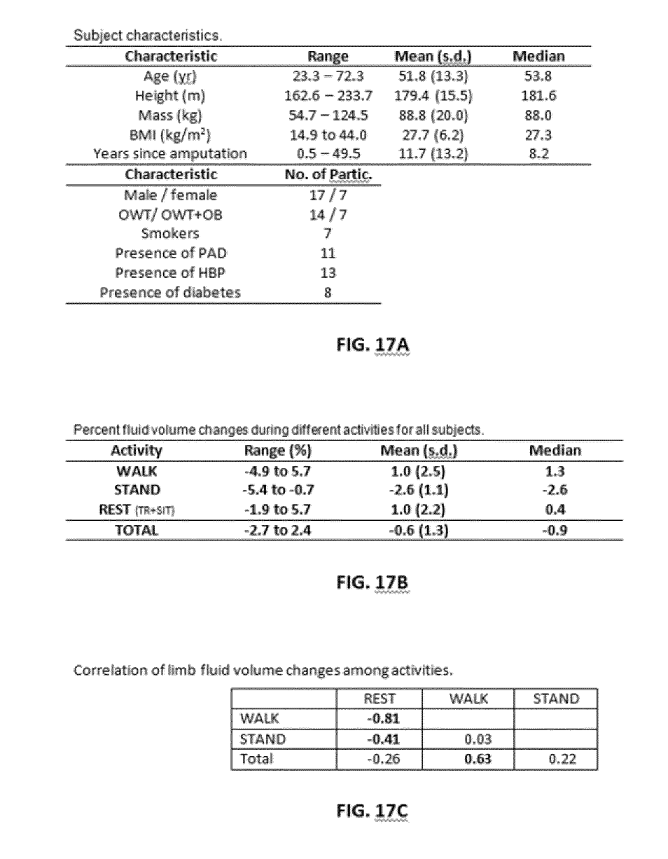

Referring now to FIG. 16, some components of an ambulatory bioimpendence system that may be included in an ambulatory controller 52 (see FIG. 1A) are shown schematically. A microcontroller provides direct digital synthesis control signals DDS to a voltage control current source VCCS, which injects a desired current into the limb. Signals are sensed at one or more sensing electrodes V1+, V1−, V2+, V2−, etc., and the signals are transmitted to the microcontroller via an analog to digital converter ADC. The microcontroller also may have one or more sensors to identify activity and/or orientation of the patient, such as a force sensing resistor FSR which may identify steps of the patient when walking, loads from standing or the like. While the exemplary FSR and bioimpedance system shown here is also suitable for use with amputees, the vascular disease identification techniques, methods, and systems described herein, including the electrodes for use therein, will often be configured for use with non-amputee subjects or patients.

+++

### Experimental Measurements

Artery-to-vein disease states of a limb (such as compartment syndrome, vascular trauma, venous insufficiency, and the like) may impact residual fluid volume of the limb. The effects of these disease states on the residual fluid volume may be more accurately detected and localized when measurements are taken before, during, and/or after changes in the residual fluid volume, such as those that may result from changes in exertion of the patient, changes in orientation of the limb, post-trauma fluid accumulation, and/or the like. Residual fluid volume is also a factor in proper fitting and use of prosthetics for amputees. More specifically, residual limb volume change can cause socket fit problems for people who use prosthetic limbs. When the residual limb reduces in volume, the socket may become too loose, leading to excessive pistoning, gait instability, and possibly a fall or injury. When the residual limb increases in volume, the socket may become tight and uncomfortable. Excessive pressures may restrict vascular inflow, denying cells of nutrients and inducing soft tissue injury.

The volume a residual limb of an amputee loses or gains depends on several factors relating to the prosthetic used with that limb. Research done in connection with the present application indicates that during 5 minutes of treadmill walking, subjects with vascular health problems may lose more fluid volume than subjects without vascular problems. To compare limb fluid volume changes during different activities (walking, standing, resting), subjects with trans-tibial amputation were tested as follows.

The human subject volunteers in this study had a trans-tibial amputation at least 6 months prior and at least 5 h/day were using a prosthetic limb. The subjects were capable of 2 minutes of continuous treadmill walking at a self-selected walking speed as well as 2 minutes of continuous standing. Residual limb lengths were at least 9 cm to facilitate separation of the electrodes on the residual limb. The subjects were tested for presence of high blood pressure (orthostatic blood pressure (OBP)) and peripheral arterial disease (segmental limb pressures, ankle-brachial index (ABI)). An electronic blood pressure measurement unit (HEM-775, Omron, Kyoto, Japan) was used for OBP testing, and a commercial segmental limb pressure measurement system (TD312 Cuff Inflator, MV10 Manifold Selector, and SC12 and SC10 cuffs, Hokanson, Bellevue, Wash.) were used for segmental limb pressures and ABI.

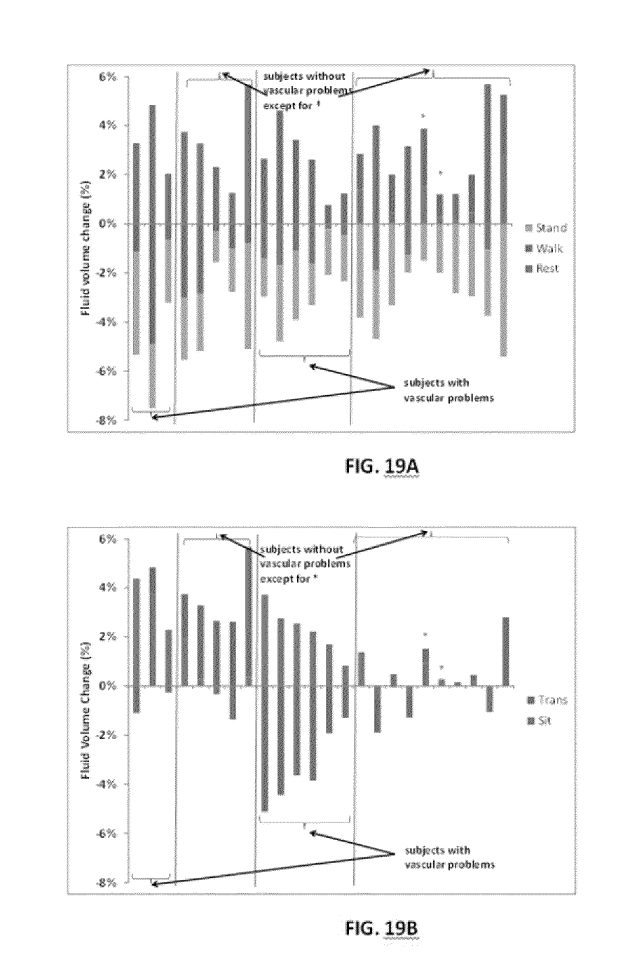

A commercial bioimpedance analyzer (XiTRON Hydra 4200, Impedimed, San Diego, Calif.) was modified to measure residual limb fluid volume as described above. Electrical current between 100 and 700 μA was injected at 50 frequencies between 5 kHz and 1 MHz through current injection electrodes on proximal and distal aspects of the residual limb. Voltage was sensed with voltage sensing electrodes positioned between the two current-injecting electrodes. The current and voltage signals were demodulated within the XiTRON unit to calculate magnitude and phase difference for each frequency. The sampling rate of the XiTRON instrument was approximately 1 Hz, and the resulting limb extracellular fluid volume measurements are shown graphically in FIG. 18A.

The results shown in FIG. 18A were obtained with 4 electrodes arranged in parallel with each other. The electrodes were made using conductive tape (ARCare 8881, Adhesives Research, Glen Rock, Pa.) (0.09 mm thickness) with an underlying hydrogel (KM10B, Katecho, Des Moines, Iowa) and a thin layer of coupling agent (ultrasonic coupling gel, Panametrics, West Chester, Ohio). Multi-stranded silver-plated copper wire (32 AWG) with an Aramid core strand and polyvinyl chloride (PVC) insulation (New England Wire, Lisbon, N.H.) (0.76 mm o.d.) was provided and connected to each electrode by sandwiching the wire between two layers of the adhesive sides of the conductive tape. Wires extending from the electrodes were strain relieved using Tegaderm (3M, St. Paul, Minn.). Outside the socket a connector with gold-plated pins (WPI Viking, Cooper Interconnect, Chelsea, Mass.) was provided and used to connect the four electrodes to a coaxial cable that attached to the XiTRON unit with a robust connector (MS3116F106S, Burndy, Manchester, N.H.). The peak-to-peak fluctuation in the bioimpedance signal while the subject stood bearing weight was less than 0.2% of the limb fluid volume.

After the subject arrived at the lab, we recorded the subject's mass and height. Then the subject sat for 10-15 minutes to achieve a homeostatic condition and answered questions about health. Sites where electrodes were to be placed were cleaned (Tracer Prep, 3M). Electrodes were placed on the limb such that all electrodes were parallel with each other. The proximal voltage sensing electrode was placed at the level of the patellar tendon on the posterior lateral surface of the limb proximal of the fibular head. The distal current injecting electrode was placed as far distally as possible but still on the cylindrical portion of the residual limb. The distal voltage sensing electrode was placed at least 3 cm proximal of the distal current injecting electrode. The proximal current injecting electrode was placed at least 7 cm proximal to the proximal voltage electrode, outside of the socket under the proximal end of the elastomeric liner or sleeve suspension of the prosthetic.

Continuous bioimpedance data collection was initiated with a sampling rate of about 1 Hz. The subject then rested in a chair for 90 s (REST). Care was taken to ensure a proper sitting posture during all rest periods. Then the subject was asked to stand with equal weight bearing on an electronic scale embedded within a short platform (STAND). Then the subject moved onto a treadmill and walked for 90 s at a self-selected walking speed (WALK). The same speed was used for all WALK cycles in a session. The subject then returned to the scale to stand under equal weight-bearing for approximately 10 s, and then sat down in the chair. The cycle of 90 s REST, 90 s STAND, 90 s WALK, and 10 s stand was then repeated four additional times. At the conclusion the subject sat down. Bioimpedance data collection was terminated and the electrodes were removed.

Demodulated data was stored and converted to extracellular and intracellular fluid impedances using a Cole model. An anatomical limb model was used to calculate extracellular fluid volume from the Cole model results and limb dimension measurements. Some subjects were adjusting to the treadmill during part of the first WALK cycle so that the first sit/stand/walk cycle was not used in the analysis. The beginning and end of each REST, STAND, and WALK phase within each of the four subsequent cycles were identified and labeled.

Fluid volume changes during each phase (REST, STAND, and WALK) of each of the last four cycles of REST/STAND/WALK were calculated. Phases for two examples cycles are shown in FIG. 18B. The REST change in each cycle was calculated as the difference in fluid volume at the beginning of the subsequent STAND from that during the previous brief stand after WALK. The STAND change was the difference in fluid volume from the beginning to end of the 90 s STAND. The WALK change was calculated as the difference in fluid volume between the brief stand after WALK and the end of the immediately prior STAND. Thus only data collected during standing with equal weight-bearing were used to calculate fluid volume changes during the three phases (REST, STAND, and WALK).

Referring now to FIG. 18B, each REST phase was partitioned into TRANSITION (stand-to-sit and sit-to-stand actions) and SIT. SIT fluid volume change was calculated as the difference in fluid volume between the end and beginning of the SIT phase. TRANSITION was defined as the SIT magnitude subtracted from the REST magnitude. Thus TRANSITION is the sum of the fluid volume change both from sitting down and from standing up. The fluid volume changes during each phase (WALK, STAND, and REST) as well as those for each part of REST (TRANSITION, SIT) were summed for each subject over the test session. All data were then referenced to the fluid volume measured during the brief stand after the first WALK, expressed as a percentage change relative to that extracellular fluid volume.

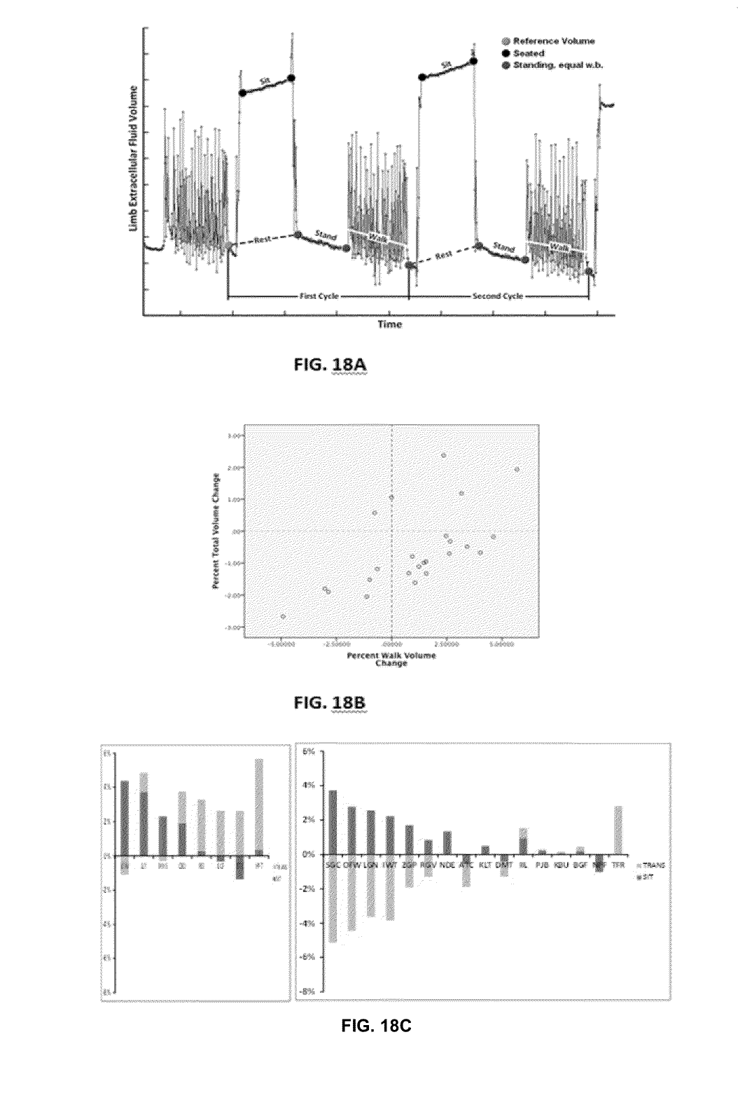

An analysis identified relationships between fluid volume changes and subject characteristics, including gender and presence of peripheral arterial disease. A total of 26 volunteers participated in the study. However, data from two subjects were not included in analysis because their residual limb lengths were outside of the calibration range acceptable for use of the bioimpedance instrument. Data from the remaining 24 subjects are presented below. Subject characteristics are listed in FIG. 17A.

Analysis of fluid volume changes over the test session for the different activities (WALK, STAND, REST) showed that the highest mean fluid volume loss was during STAND, averaging a 2.6% (s.d. 1.1%) loss over the test session, as can be seen in FIG. 17B. On average, subjects gained fluid volume during WALK and REST, with a mean limb fluid volume increase of 1.0% (s.d. 2.5%) during WALK and 1.0% (s.d. 2.2%) during REST. Fluid volume changes during STAND were significantly different from those during WALK (p<0.001), and from those during REST (p<0.001). All participants lost fluid volume during STAND. The rate of fluid volume loss within the STAND periods was calculated to show an average rate of change of −0.43%/minute (s.d. 0.18). FIG. 17C shows the correlations among the various activities and the percent total volume change (bold face represents statistically significant results at 0.05 level or smaller). There were statistically significant differences in distributions of percent volume change between individuals with and without HBP for STAND (p=0.015), where loss of volume was larger among people without HBP (median=−2.8) when compared to people with HBP (median=−1.9). Inspection of the data for the eight subjects who lost fluid volume during WALK revealed differences for subjects with and without PAD.

During the REST period, subjects with peripheral arterial disease may transport fluid slowly into the residual limb compared to subjects without PAD who may transport fluid quickly into the residual limb. Thus subjects with PAD may have low TRANSITION fluid volume gains and high SIT fluid volume gains. Subjects without peripheral arterial disease may have high TRANSITION fluid volume gains and low SIT fluid volume gains. Data consistent with these mechanisms are shown in FIG. 18C. The four subjects with PAD had high SIT values and low TRANSITION values, while subjects without PAD had high TRANSITION values and low SIT values. Six of eight subjects with PAD who gained fluid volume during WALK experienced high SIT values and low TRANSITION values, similar to PAD subjects who lost fluid volume during WALK. However, losses during TRANSITION were much greater for these six subjects compared with the four subjects with PAD who lost fluid volume during WALK.

While one might expect subjects with vascular co-morbidities to be the only subjects to demonstrate fluid volume losses during WALK, we found that only half of the subjects who lost fluid volume during WALK had PAD (four of eight (including one subject on lifetime antibiotics)). Those four subjects who lost fluid volume during WALK but were healthy, however, did show one notable difference compared to the four subjects with PAD. They quickly moved fluid into and out of the residual limb during REST, unlike PAD subjects.

Though most participants compensated for the fluid volume loss during STAND by increasing fluid volume gain during WALK, some of them experienced so much fluid volume loss during REST that their overall fluid volume change was negative. Most of these participants had PAD as shown by the graph in FIG. 18C. Interestingly, these subjects lost considerable volume during TRANSITION, not during SIT. This result suggests that fluid gained during SIT was easily displaced out of the limb upon rising for the subsequent STAND. Because of the quick expulsion upon standing, we suspect the fluid was primarily blood within the arteries and veins. These subjects appeared to have impeded fluid transport across the arterial walls into the interstitial space (consistent with their PAD), or very rapid transport from the interstitial space into the venous vasculature, or both. The two subjects with PAD who were outliers in that they were fast transporters (i.e. had high TRANSITION-SIT values) may have also had venous insufficiency, offsetting the reduced arterial-to-interstitial transport induced by presence of their PAD. If they did have venous insufficiency then the result for them would be a better balance between fluid entering and fluid leaving their residual limb compared to other subjects in the study, albeit with both likely at a reduced flow rate compared with healthy subjects. Venous insufficiency often is not a systemic problem but instead a local one. A subject might not demonstrate presence of venous insufficiency in the contralateral limb using standard test methods but may still have venous insufficiency in the residual limb.

Most of the participants' fluid volumes changes over the test session were dominated by differences between their STAND and WALK fluid volume changes (right side of figure). It is worth noting that our sitting interval in this protocol was only 90 s long. Additionally, subjects with high TRANSITION values were mainly women. Women, in general, may not empty their veins as rapidly as men, so when socket pressures are released (e.g., transitioning from standing to sitting) arterial and interstitial fluid levels may increase dramatically within the residual limb. This change would happen because of the slow capability to empty the veins limiting limb fluid outflow, and thus may explain why subjects with high TRANSITION values were mainly women. The single female subject who did not demonstrate these trends was the only female subject who had PAD. Possibly her arterial occlusion offset her limited venous outflow so that she maintained good limb fluid balance.

Additional graphical data from amputee subject testing is shown in FIGS. 19A and 19B. These results indicate that subjects with vascular problems, in general, can be delineated from subjects without vascular problems by examination of their fluid volume changes via bioimpedance data gathered using the methodology and systems described herein during sitting, standing, walking, and transition activities.

A number of variations and modifications of the disclosed embodiments can also be used. Specific details are given in the above description to provide a thorough understanding of the embodiments. However, it is understood that the embodiments may be practiced without these specific details. For example, well-known circuits, processes, algorithms, structures, and techniques may be shown without unnecessary detail in order to avoid obscuring the embodiments.

Implementation of the techniques, blocks, steps and means described above may be done in various ways. For example, these techniques, blocks, steps and means may be implemented in hardware, software, or a combination thereof. For a hardware implementation, the processing units may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments may be described as a process which is depicted as a flowchart, a flow diagram, a swim diagram, a data flow diagram, a structure diagram, or a block diagram. Although a depiction may describe the operations as a sequential process, many of the operations can be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks may be stored in a machine readable medium such as a storage medium. A code segment or machine-executable instruction may represent a procedure, a function, a subprogram, a program, a routine, a subroutine, a module, a software package, a script, a class, or any combination of instructions, data structures, and/or program statements. A code segment may be coupled to another code segment or a hardware circuit by passing and/or receiving information, data, arguments, parameters, and/or memory contents. Information, arguments, parameters, data, etc. may be passed, forwarded, or transmitted via any suitable means including memory sharing, message passing, token passing, network transmission, etc.

For a firmware and/or software implementation, the methodologies may be implemented with modules (e.g., procedures, functions, and so on) that perform the functions described herein. Any machine-readable medium tangibly embodying instructions may be used in implementing the methodologies described herein. For example, software codes may be stored in a memory. Memory may be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” may represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A method for identifying a potential vascular disease state of a limb of a patient, the limb comprising an outer surface and a first region, the method comprising:
placing a plurality of electrodes on the limb, including a first electrode on the outer surface of the limb adjacent the first region;
energizing at least some of the electrodes while the limb of the patient is in a first arterial to vein transport state so as to generate a first signal with the first electrode;
determining that the first signal is consistent with the potential vascular disease state of the limb in response to the first signal; and
providing an indication of the potential vascular disease state of the limb.

2. The method recited in claim 1, the first region of the limb including tissue with associated veins and arteries, further comprising:
placing a second electrode pair on the outer surface of the limb adjacent a second region of the limb, the second region including tissue with associated veins and arteries; and
generate a second signal with the second electrode; and
determining the first signal and the second signal correspond to the potential vascular disease state, the potential vascular disease state comprising an artery-to-vein disease state.

3. The method recited in claim 2, further comprising:
localizing the potential vascular disease state in the first region in response to the first and second signals; and
selectively directing therapy to the first region so as to mitigate the potential vascular disease state.

4. The method of claim 3, the tissue of the first region comprising muscle tissue of a first muscle compartment of the limb, the tissue of the second region comprising tissue of a second muscle compartment of the limb, further comprising performing fasciotomy to the muscle tissue within the first muscle compartment and foregoing fasciotomy to the muscle tissue within the second muscle compartment in response to the localization of the potential vascular disease state within the first muscle compartment.

5. The method recited in claim 1, further comprising:
storing information from the first signal relating to the state of the region of the first muscle of the limb and energizing the at least some electrodes while the limb of the patient is in a second arterial to vein transport state so as to generate a second signal, wherein the first signal is generated at a first time and the second signal is generated at a second time; and
determining that a trend between the first signal and the second signal is consistent with a potential artery-to-vein disease state of the limb.

6. The method recited in claim 5, wherein the first electrode pair is included in an ambulatory bioimpedance system mounted to the patient during the first and second times.

7. The method recited in claim 6, wherein the first artery-to-vein transport state comprises a relatively active exertion state and the second artery-to-vein transport state comprises a relatively inactive exertion state.

8. The method recited in claim 6, wherein the first artery-to-vein transport state comprises a first orientation of the patient and the second artery-to-vein transport state comprises a different orientation of the patient.

9. The method recited in claim 6, wherein the limb comprises a leg and the patient is walking or running during the first time and is resting during the second time.

10. The method recited in claim 6, wherein the second state occurs after the first state and the trend is associated with post-exertion recovery.

11. The method recited in claim 6, further comprising supporting the ambulatory bioimpedance system on the patient for over 1 hour and identifying the potential disease state using a plurality of trends between differing artery-to-vein transport states of the limb.

12. The method recited in claim 5, wherein:
the placing of the electrode pair is performed in a field setting and the first time is within 2 hours of a trauma to the patient, and the second time is after the first time by a post-trauma fluid accumulation period.

13. The method recited in claim 1, wherein the electrodes are included in a bioimpedance system and are configured for mounting to the limb with each electrode in an associated limb location, and wherein the bioimpedance system is used to perform the energizing of the at least some electrodes, the bioimpedance system applying a plurality of frequencies to the at least some electrodes so as to generate a plurality of signals and determining the disease state from the plurality of signals.

14. A system configured to identify a potential vascular disease state of a limb of a patient, the system comprising:
a plurality of electrodes including a first limb surface electrode;
a controller configured to:
energize at least some of the electrodes so as to generate a first signal with the first electrode;
determine that the first signal is consistent with a potential vascular disease state of the limb in response to the first signal; and
provide an indication of the potential disease state of the limb.

15. The system recited in claim 14, wherein the plurality of electrodes includes a second limb surface electrode and a current injecting electrode, wherein the controller is further configured to send and receive signals from the first electrode via a first communication channel and to send and receive signals from the second electrode via a second communication channel, to energize the second electrode so as to generate a second signal, and to determine if the second signal is consistent with a potential artery-to-vein transport disease state in response to the second signal.

16. The system recited in claim 15, wherein the controller is configured to provide an indication of a location of the potential disease state of the limb in response to the first and second signals.

17. The system recited in claim 14, wherein the controller is configured to energize the at least some electrodes so as to generate the first signal by energizing the first electrode at a first time.

18. The system configured to identify a potential disease state of a limb of a patient recited in claim 17, wherein the controller is further configured to store the first signal.

19. The system recited in claim 18, wherein the controller is further configured to energize the at least some electrodes at a second time so as to generate a second signal.

20. The system recited in claim 19, wherein the controller is further configured to store the second signal.

21. The system recited in claim 19, wherein the controller is further configured to determine that a trend between first signal and the second signal is consistent with a potential artery-to-vein transport disease state.

22. The system recited in claim 19, wherein the system comprises an ambulatory system and at least a portion of the controller is included in an ambulatory controller body having mount for affixing the ambulatory controller to the patient.

23. An apparatus for use in a system configured to identify a potential vascular disease state of a limb of a patient, the limb comprising a first tissue, the system including the apparatus and an ambulatory controller having an plurality of electrode interfaces and configured to provide an energizing signal to at least some of the interfaces and to determine that a first signal at a first of the interfaces is consistent with a potential vascular disease state of the limb, the apparatus comprising:
a set of electrodes configured for mounting to the limb, each electrode having an interface configured to couple to the interface of the controller so that at least some of the electrodes are energized by the energizing signal;
wherein a first electrode of the set of electrodes is configured to be mounted to a limb surface of the limb so as to generate the first signal.
